(19) United States
(12) Patent Application Publication
(10) Pub. No.: US 2005/0228313 A1
(43) Pub. Date: Oct. 13, 2005

(54) Fluid sampling, analysis and delivery system

(75) Inventors: Karan V.I.S. Kaler, Bragg Creek (CA); Giorgio Gattiker, Calgary (CA); Martin P. Mintchev, Calgary (CA)

Correspondence Address: THOMPSON LAMBERT;SUITE 703D, CRYSTAL PARK TWO, 2121 CRYSTAL DRIVE, ARLINGTON, VA, 22202, US

(73) Assignee: University Technologies International Inc., Calgary (CA)

(21) Appl. No.: 11/003,027

(22) Filed: Dec. 3, 2004

Related U.S. Application Data
(60) Provisional application No. 60/526,595, filed on Dec. 4, 2003.

(51) Int. Cl. A61M 1/00; A61B 5/00; B65D 81/00

(52) U.S. Cl. 600/583; 600/573; 604/27

(57) ABSTRACT

The lack of safe, reliable, automated and clinically acceptable blood sampling has been the main problem precluding the development of real-time systems for blood analysis and subsequent closed-loop physiological function control. While the analysis of a static blood sample in laboratory conditions has been rapidly advancing in reliability and blood volume reduction, non-invasive real-time blood analysis performed in vivo (while the blood is circulating in the body) has been elusive and unreliable. In this study we propose an innovative idea for semi-invasive blood sampling and analysis, which resembles the operation of a mosquito. At a miniature scale the proposed system does penetrate the skin to extract a static blood sample for further analysis, but the extent of this penetration, and the fact that it can be made painless, is particularly attractive for such applications as automated glucose analysis for closed-loop control of insulin infusion (artificial pancreas), continuous drug monitoring, or even periodic DNA analysis for security and identification purposes. These design aspects are described, and a specific implementation, applying MEMS (Micro Electro Mechanical Systems) technology, is suggested. The proposed microsystem is a matrix of individually controllable e-Mosquito™ cells, packaged in a disposable patch and attached to the skin, could be an avenue for real-time semi-invasive blood analysis and diagnostics.

### CROSS-REFERENCE TO RELATED APPLICATIONS

This application claims the benefit of priority under 35 U.S.C. 119(e) of U.S. provisional application No. 60/526,595 filed Dec. 4, 2003.

### BACKGROUND OF THE INVENTION

Blood sampling is essential for diagnosing and testing a wide variety of disorders and medical conditions, as well as for DNA testing, and blood donation screening [1]. Contemporary blood sampling techniques can be generally classified as invasive, implanted and non-invasive [2]. All references in square brackets are listed at the end of the patent disclosure and incorporated by reference herein.

Non-invasive blood sampling and subsequent analysis usually involves optical or ultrasonic tissue interrogation techniques [3], which generally depend on the location and the characteristics of the tissue volume studied. For example, it has been shown that glucose concentration in a given blood volume does have specific optical and ultrasonic signatures [4], and claims have been made that interrogation of the optical or the ultrasonic spectra absorbed or reflected by a tissue volume rich in capillaries could be indicative of glucose concentration dynamics [5]. However, the impact of non-quantifiable external factors (e.g., variable capillary concentration in the interrogated tissue volume, changeable capillary volumes associated with blood pressure dynamics, dynamic absorption capabilities of the surrounding living tissue, etc.), make the reliability, the repeatability and the associated errors of non-invasive blood sampling techniques inadequate to warrant wide-spread and routine utilization [6].

Implantable blood sampling involves the permanent or temporary introduction of a blood-monitoring enclosure inside the body. A variety of electrochemical techniques have been suggested, but the limitations of this approach include foreign-body reactions related to scar tissue buildup around the sensor, and the complexities associated with data transmission from the implant to external data loggers. In important studies, inconsistently decreasing sensor activity has been found in less than 24 hours, questioning the repeatability of this approach even if the signal transmission issues were to be completely resolved [2, 7-9].

To this day, invasive blood sampling techniques remain the most reliable and routinely utilized approach in the clinical practice [1]. Dramatic recent developments in miniaturizing the sampling needles [10-19] and the volume of blood needed for reliable analysis [20] have significantly facilitated this centuries-old approach, and contemporary commercially-available blood monitors have the convenient miniature size of cellular telephones [21, 22]. Nevertheless, needle based blood collection is necessary in order to harvest an adequate volume of blood for analysis and electronic reporting, which presents a significant inconvenience for wide groups of patients and medical professionals alike, such as diabetic patients and laboratory nurses. The risk of infections, and the associated pain and skin irritations should not be underestimated as well.

Many intellectual property instruments (patents and patent applications) have been released on invasive blood sampling [21, 23-29] and on transdermal drug delivery through microneedles [23, 30-32], however these approaches are deficient in automated real-time actuation and control of the microneedle. The skin penetration as well as the extraction of the needle is hence executed manually.

Recent technological improvements in the area of micro-electromechanical systems (MEMS) significantly facilitated the development of automated, invasive blood sampling and drug delivery devices. Some references [33, 34] describe an autonomous, ambulatory analyte monitor or drug delivery device [33] or a Microneedle Transdermal Transport Device [34]. These approaches do not present a self-contained, real-time, closed-loop control device, utilizing an integrated microsystem mountable on a disposable adhesive patch, and containing a matrix of individually, but sequentially actuated, single-use elementary cells mounted in a disposable platform such as a self-adhesive patch.

### SUMMARY OF THE INVENTION

The aims of the present study and patent disclosure are: (1) to suggest a conceptually new, fourth approach in blood sampling, which could be characterized as semi-invasive and is based on a device defined as the Electronic Mosquito or e-Mosquito™; (2) to outline, model and prove feasibility of the e-Mosquito™ building blocks; (3) to present comprehensively the integrated e-Mosquito™ system and demonstrate its principle of operation; (4) to discuss fabrication-related issues related to the proposed design; and (5) to evaluate the capabilities and the limitations of this new approach in blood sampling, glucose monitoring and drug delivery.

The principle of semi-invasive blood sampling could be easily related to the operation of a mosquito, which penetrates the skin to collect a very small volume of blood, with minimal amount of skin irritation. Smooth penetration is ensured because of the jagged shape of the maxilla, and blood transport over relatively long distance is made possible by a feeding pump. Anesthetizing saliva provides for a painless penetration, while the minimal irritation is associated with the anticoagulant used [35] (FIG. 3.1a). The electronic cousin of the mosquito, the e-Mosquito™, somewhat differs from the approach utilized by the real mosquito, although the end result, the acquisition of a blood sample, remains the same (FIG. 3.1b).

The total size of one single e-Mosquito™ may be in the range of a few millimeters and a set of these miniature cells can be applied as a patch on to the skin, similarly to a band-aid. The short microneedle provides completely painless blood sampling. The electrochemical sensors need only a very small blood sample for reliable assessment of blood glucose or other blood parameters, utilizing the collected static blood volume. An antiseptic layer provides minimal risk of infection and skin irritation. The e-Mosquito™ is a single-use device. The device features a real-time monitoring of blood parameters and is capable of sending wirelessly the results either to a remote device with a display (i.e. wrist watch, cell phone, personal digital assistant, etc.) or directly to a medical authority (i.e. hospital, clinic, medical doctor, etc.)

Taking into account the technological complications related to designing a microelectronic “horizontal drilling” setup to mimic the operation performed by the maxilla of the real mosquito (see FIG. 3.1a), a simple vertical penetration was considered. However, the “horizontal drilling” of the mosquito has several important biological advantages, including the possibility to search for arteries or capillaries, and the capability to directly penetrate a vessel, thus extracting maximal blood volume from it. In the case of the e-Mosquito™, however, both of these advantages can be bypassed by (a) introducing a matrix of single-use e-Mosquito™ cells (FIG. 3.2), with the individual actuation of which a direct vertical hit on a capillary by one of the needles becomes inevitable; and (b) reducing the blood volume needed for reliable analysis.

The second major difference between an actual mosquito and its “electronic cousin” is the penetrating depth, which in the latter case is about 400 um, thus ensuring the reach of capillaries but not neural endings, thus providing for a painless operation (FIG. 4.1), and also avoiding the need for accompanying anesthetizing liquid during the bite.

Thirdly, the e-Mosquito™ does not have a feeding pump, but relies on the intricate balance between capillary forces and pressure differences considered during the design process. This intricate balance, combined with the fact that each e-Mosquito™ cell in the matrix is a single-use device, also avoids the use of anticoagulant and alleviates the need to clean individual cells after use, thus keeping the microneedle actuation a sterile one-time event. In other words, each individual e-mosquito cell within the framework of the matrix dies immediately after fulfilling its primary mission, which is to extract a given volume of blood required for reliable stationary blood analysis associated with the particular application of the device. Should a needle from a given cell of the matrix fail to hit a capillary and fulfill its mission, the entire cell is not reused—another cell picks up the mission at a slightly different location.

In addition, penetration of the skin and microneedle withdrawal in the e-Mosquito™ is performed through an antiseptic layer adhesive to the skin, thus avoiding any potential skin irritations or infections.

A new concept of an integrated and automated microsystem for blood sampling, multicomponent blood monitoring and analysis, and drug delivery is presented. Important design parameters have been laid out and quantified to demonstrate the capabilities and limitations of the e-Mosquito™ patch. The building blocks of the e-Mosquito™ cell are outlined, modeled and shown to be feasible to implement. The integrated e-Mosquito™ system is presented comprehensively and its principle of operation is demonstrated illustratively. A realistic fabrication process is described for smooth implementation of the proposed e-Mosquito™ design. Further summary of aspects of the invention is found in the claims, which are incorporated by reference here.

### BRIEF DESCRIPTION OF THE FIGURES

There will now be described preferred embodiments of the invention by way of illustration with reference to the figures, in which the figures listed on the left show the item described on the right in the following:

FIG. 3.1a The Mosquito.

FIG. 3.1b The e-Mosquito™.

FIG. 3.2 The e-Mosquito™ patch.

FIG. 4.1 Layers of the skin and painless microneedle penetration.

FIG. 4.2 Proposed silicon microneedle.

FIG. 4.3 Fluid mechanic phenomena in a circular needle channel.

FIG. 4.4a Actuation principle of a piezoelectric bimorph beam.

FIG. 4.4b Dimensions of the piezoelectric heterogeneous bimorph beam.

FIG. 4.5 Silicon microactuator structure.

FIG. 4.6 ANSYS-based Finite Element Analysis of a Silicon microbridge.

FIG. 4.7 Induced current and blood glucose concentration relationship.

FIG. 4.8 The e-Mosquito™ microsensor structure.

FIG. 4.9 The e-Mosquito™ electrical block diagram.

FIG. 4.10 Individual multiplexed control of a single e-Mosquito™ cell.

FIG. 4.11 Control of a single e-Mosquito™ drug delivery cell.

FIG. 5.1 Exploded 3D view of the e-Mosquito™ building blocks.

FIG. 5.2 Compact 3D view of the e-Mosquito™ building blocks.

FIG. 5.3 2D view of a FEA applied to the e-Mosquito™ assembly.

FIG. 5.4 3D view of a FEA applied to the e-Mosquito™ assembly.

FIG. 5.5 3D top view of the e-Mosquito™ microactuator structure.

FIG. 5.6 3D view of the e-Mosquito™ microsensor structure.

FIG. 5.7 The e-Mosquito™ blood sampling process.

FIG. 5.8 Integration of the single cell into the e-Mosquito™ matrix.

FIG. 5.9 3D bottom view of the e-Mosquito™ matrix.

FIG. 5.10 Exploded 3D view of the e-Mosquito™ patch.

FIG. 5.11 Compact 3D view of the e-Mosquito™ patch.

FIG. 5.12 Attachment of the e-Mosquito™ patch to the shoulder of a subject.

FIG. 6.1 Fabrication steps of the e-Mosquito™ PZT microactuator.

FIG. 6.2 Fabrication steps of the microactuator and the microbridge.

FIG. 6.3 Fabrication steps of the microbridge and the microneedle.

FIG. 6.4 Fabrication steps of the e-Mosquito™ microsensor structure.

FIG. 6.5 Fabrication steps of the e-Mosquito™ microsensor electrodes.

FIG. 6.6 Fabrication steps of the e-Mosquito™ microelectronics.

FIG. 6.7 The individual fabricated e-Mosquito™ building blocks.

FIG. 6.8 The bonded e-Mosquito™ building blocks.

### DETAILED DESCRIPTION OF PREFERRED EMBODIMENTS OF THE INVENTION

Design of the Building Blocks

Microneedle

A variety of different approaches have been put forward in designing the microneedles. In terms of fabrication techniques, in-plane [13, 17, 20, 36, 37] and out-of-plane [11, 12, 15, 16, 38-46] solutions have been suggested. Single microneedles are normally produced in-plane, and are much larger in size, while out-of-plane designs are smaller and are routinely fabricated in a matrix. A clear distinction is made between microneedles for drug delivery, and for blood sampling. In-plane needles are efficiently used for blood sampling because of their longer size [13, 20, 36]. With the recognition of the efficacy of subcutaneous drug delivery, out-of-plane microneedles are routinely preferred for that purpose [12, 15, 38, 47].

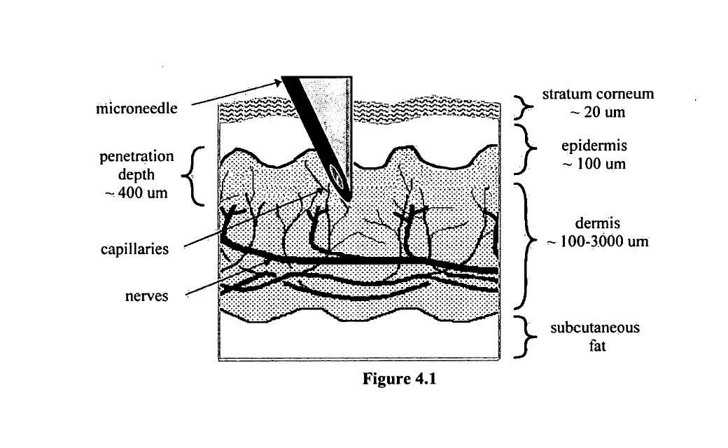

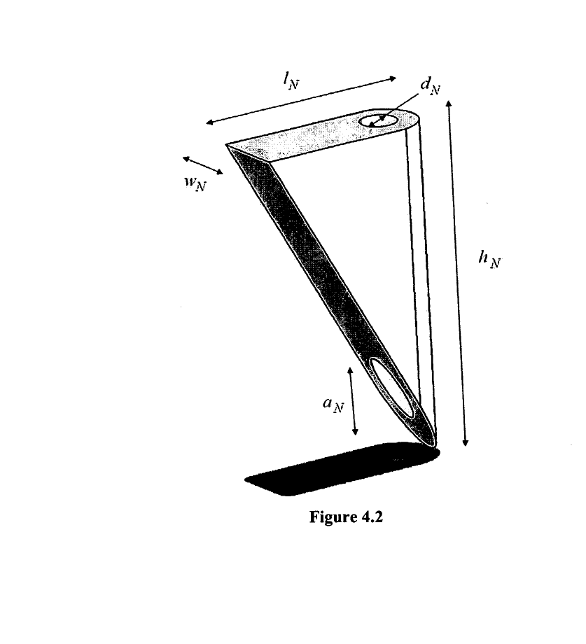

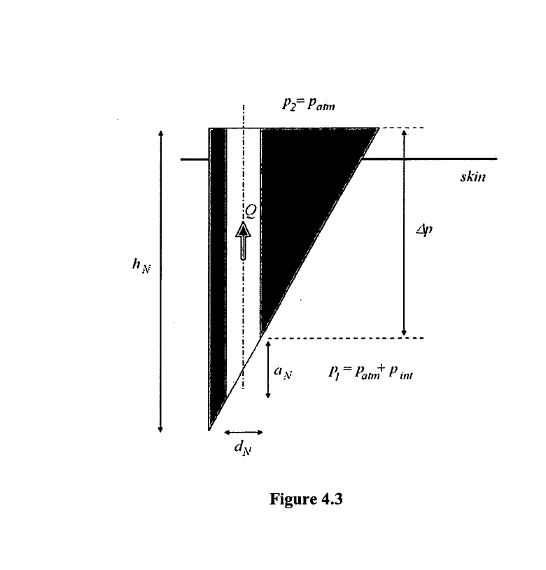

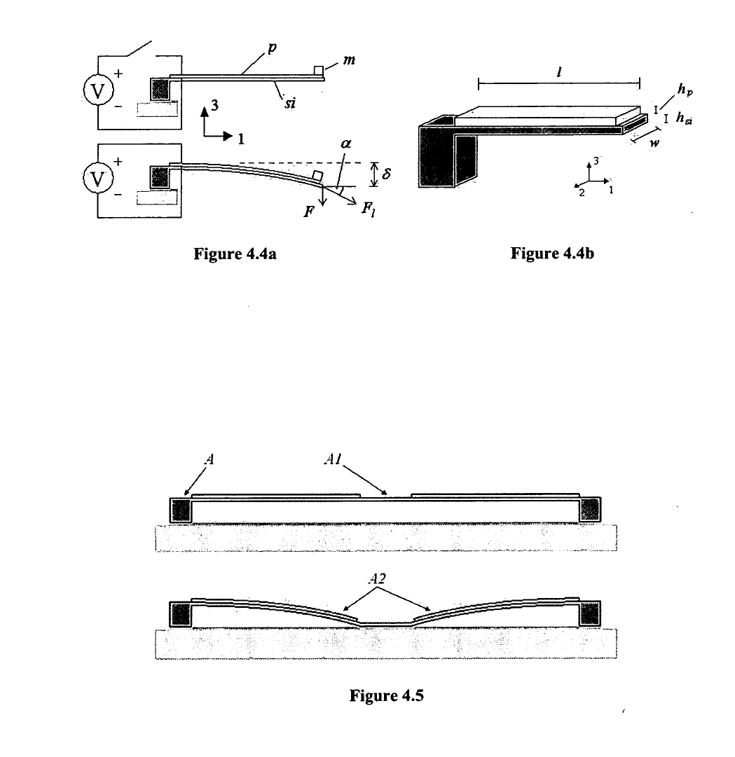

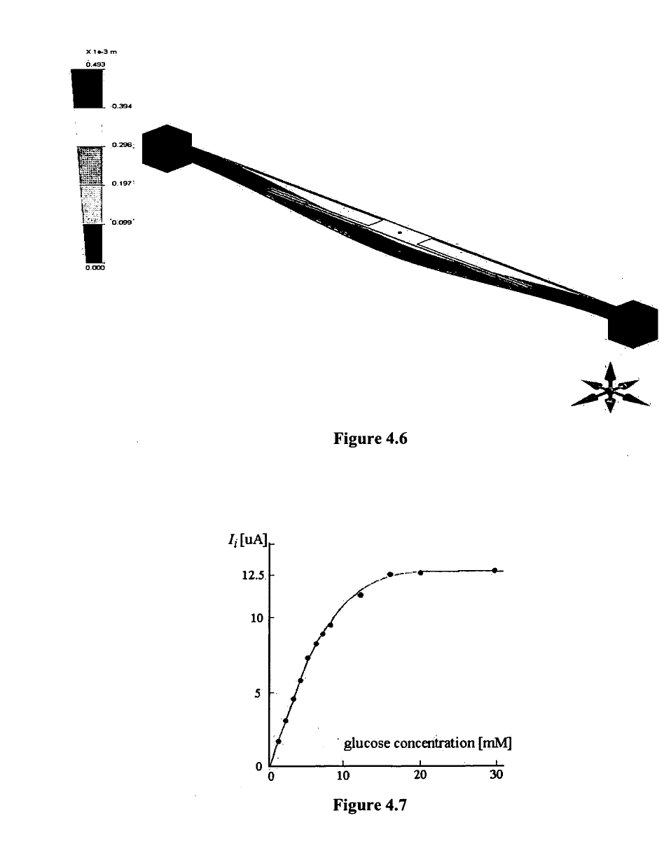

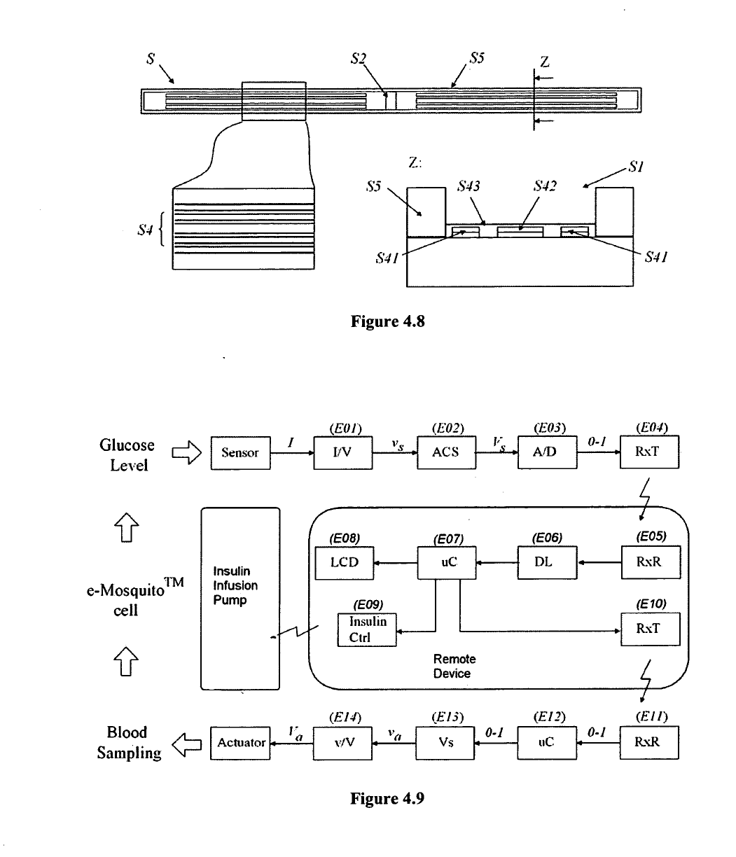

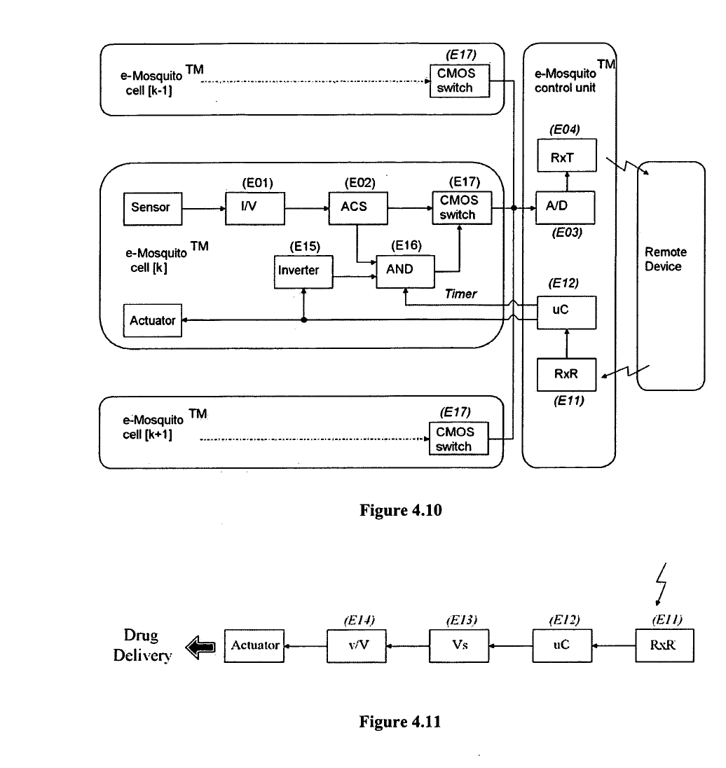

The human skin is composed of three layers: (1) the stratum corneum is the outermost layer and is made of dead tissues; (2) the epidermis, a tissue of living cells and interstitial plasma, which has a thickness of about 100 um; and (3) the underlying dermis with blood capillaries and nerves [18]. The ultimate goal is to reach the blood vessels without affecting the nerves. In the specific microneedle design for the e-Mosquito™, optimal painless penetration depth of about 400 um for minimally invasive liquid transfer was considered [11, 16, 38, 40] (FIG. 4.1).

Opening a bracket in the overall design discussion, it should be mentioned that the e-Mosquito™ design was implemented keeping in mind potential drug-delivery applications as well. Considering that the pressure needed for routine skin penetration was reported to be 3.138 N/mm² [47], in the worst case scenario of a needle surface area of 100 um×300 um (0.03 mm²) penetrating the skin up to its base, the required penetration force was estimated to be up to 100 mN. This value represents the maximal force, which the microactuator has to be able to exert in a controlled manner.

Among the various silicon microneedle shapes, the out-of-plane pyramidal side-opened design [16] has been considered beneficial in terms of ease of fabrication, mechanical strength, and avoidance of clogging at the needle tip. Tests on the mentioned design have further shown that blood-like liquids are readily drawn and transported into the needle by capillary forces, thus eliminating the need for any active pumping. Such hollow needles may also be used for transdermal liquid transfer, i.e. for either blood extraction or drug delivery.

The proposed needle design differs only slightly in geometry from the reported pyramidal microneedle [11, 16] and preserves the beneficial characteristics such as high mechanical strength and enhanced fluid mechanics. However, since the microneedle is to be integrated on a microstructure, the fabrication process differs and will be discussed later in this report.

The e-Mosquito™ microneedle (FIG. 4.2) is designed to either sample blood or subcutaneously deliver medication. In order to easily break the stratum corneum, the needle has to be sharp. However, since the mechanical properties of human skin are not sufficiently well characterized or known, precise requirements for the sharpness of the needle are difficult to formulate [48].

There are two possible failure scenarios for the needle: fracture or buckling. Both could occur during the insertion of the needle into the skin. Failure may occur under load, causing either fracture or buckling, whichever is lower. A worst-case estimate of the maximum load, which the needle can withstand can be calculated assuming that the breakage is confined to a region near the microneedle tip. The fracture load can be estimated from:
P_{fr}=σ_{y}A (1)
where P_{fr} is the critical load, A is the cross-sectional area of the needle, and σ_{y} (=7 GPa) is the yield stress of single crystal silicon [49]. Since silicon is not a ductile material, the yield stress is approximately equal to the fracture stress. For this particular needle size, the estimate of fracture force in the region of the orifice at the needle tip is about 6 N, indicating that the microneedle would not break if adequately designed. Failure due to buckling is even less likely to occur, since the shape of the needle is not thin and long, but thick and pyramidal. The thicker the needle, the higher the force required to buckle it [36].

The overall microfluidic characteristics are considered next, while two scenarios of fluid flow through the microneedle are taken into account: (1) during the sampling mode, the blood flows from the needle tip up to the base; and (2) during drug delivery, the fluid flows from the base of the microneedle down towards its tip.

For the blood sampling mode two parameters are important: the pressure difference between the base and the tip, and the capillary forces. While the needle is being inserted into the skin (FIG. 4.3), the pressure p₁ at the tip is additive to the interstitial blood pressure pᵢₙₜ, which is assumed to be around 25 mmHg [50]. Since the atmospheric pressure pₐₜₘ (˜760 mmHg) is present at both ends, the pressure is higher at the tip which makes the blood flow towards the base.

To calculate the pressure-flow characteristics in the needle channel, the following relationships and assumptions are considered. For a pipe with a circular cross section, assuming laminar flow and a Newtonian fluid (incompressible fluid with constant viscosity), the flow Q is given by the Hagen-Poiseuille equation,

$$Q = \frac{π {D}^{4}}{128 µL} Δ p \qquad ( 2 )$$

where D is the channel diameter, L is the length of the channel, μ is the viscosity and Δp is the pressure difference [51]. It has to be mentioned, that although blood is generally not a Newtonian fluid it can be regarded as one in the present microfluidic calculations [51]. Substituting specific values into Eq. (2) a flow rate of Q=0.341 ul/s was obtained considering the pressure difference Δp mentioned above. This value implies that the microneedle has to be inserted into the skin for about 4 s to obtain 1 ul of blood, which is approximately the volume needed for an accurate blood glucose sensing using a standard commercially-available technology for the analysis of a static miniature blood drop [22].

Examining the mode of drug delivery, the pressure at the base of the needle p₂ has to be higher then the interstitial blood pressure pᵢₙₜ to obtain a fluid flow in the opposite direction towards the blood capillaries. Fluidic resistance is an important parameter for the characterization of flow through a channel. It is defined as the ratio of pressure drop Δp over flow rate Q [51] and has to be kept as low as possible for an efficient liquid transfer through the microneedle [41]. A circular cross section, as utilized in the present needle design, exhibits lower resistance then any other type of cross section. The radius of the circular cross section can be related to the average diameter of a red blood cell, which is about 7.5 um [50], restricting the minimal value for the diameter of the microneedle channel. Another limitation when dimensioning a microchannel is the problem of clogging. Generally, it can be assumed, that the smaller the cross-sectional area, the higher the possibility for and the velocity of clogging. Tests on microneedles confirm this assumption and it has been further shown, that a diameter in the range of 50 um is adequate to avoid blood clogging [11, 41].

For a small flow channel, the surface tension force (the capillary force) tends to draw liquid into the channel. The capillary force F_{c} for a round channel with a diameter D is [51]:
F_{c}=Dπγ cos (θ) (2)
where γ is the interfacial surface tension and θ is the contact angle between the liquid and the surface. For a vertical channel, the gravitational acceleration g on the rising column of liquid with density ρ and height h, opposes the capillary force F_{g}, and is given by:

$${F}_{g} = \frac{D π h ρ g}{4} \qquad ( 3 )$$

Equating these two forces gives the maximum rise of a fluid level h against the gravity:

$$h = \frac{4 γ cos ( θ )}{D ρ g} \qquad ( 4 )$$

In the proposed microneedle channel with a diameter D of 50 um, the blood reaches a height of 57 mm, which is more than enough to extract the blood up from the capillaries into the blood compartment of the e-Mosquito™. In previously designed out-of-plane microneedles with a similar channel radius, liquid presented to the needle base without applying a pressure difference between the two orifices was sucked into the needle channel by capillary forces [11].

Microactuator

The development of MEMS microactuators is still in its infancy mainly because of the initial lack of appropriate applications and the difficulty of reliably relating microactuators to the macroscopic world [52]. Although the earliest microactuators utilized electrostatic forces, devices now exist that are actuated by thermally-controlled shape-memory alloys (SMA), magnetic or piezoelectric forces, just to name a few [53]. Each method has its own advantages, disadvantages and an appropriate set of applications.

When choosing an actuator, the importance of the needed functionality cannot be overemphasized. The most important properties and characteristics that have to be taken into account are the complexity of fabrication, force, pressure, displacement, power consumption, voltage supply, current supply, size, precision, timing, shape and strain. The microactuator for the e-Mosquito™ has to meet at least the following estimated requirements to successfully penetrate the human skin for blood sampling. A large displacement of about 400 um is necessary to introduce the microneedle into its optimal depth without producing pain. A maximal force in the order of 100 mN is required to pierce the upper layers of the skin and to reach the blood capillaries with the specific microneedle design (see Section 4.1). In addition, the actuator should be driven with a low operating voltage in order to be compatible with contemporary microelectronic circuits, and should consume as little power as possible. Since the actuation takes place in a conductive environment, neither the performance of the microactuator, nor the blood parameters should be affected by the type of actuation. Therefore, the actuator has to be biocompatible in conductive fluids. The complexity of fabrication, another very important criterion, should be maintained as low as possible, aiming for a simple and elegant design.

Quantitative investigation of existing actuation types revealed that piezoelectric actuation is favorable in terms of force delivery capability, low complexity, fabrication feasibility, and power efficiency [51-53]. Piezoelectric materials exhibit a change in deformation when an electric field is applied to them. FIG. 4.4(a) illustrates the actuation principle of a silicon bender coated on top with a piezoelectric material. Constituent equations of piezoelectric heterogeneous bimorphs have been used to analyze the geometry of cantilevers coated with piezoelectric materials [54]. The vertical displacement δ and the slope α at the tip of a free bender with a mass m and length l can be calculated as follows:

$$\begin{matrix}δ = A [ ( \frac{4 {l}^{3} m g}{K w} ) + ( \frac{3 {d}_{31} B {l}^{2} V}{K} ) ] & ( 5 ) \\ α = A [ ( \frac{6 {l}^{2} m g}{K w} ) + ( \frac{6 {d}_{31} B l V}{K} ) ] & ( 6 )\end{matrix}$$

with the substitutions:

$$\begin{matrix}A = {s}_{11}^{s i} {s}_{11}^{p} ( {s}_{11}^{p} {h}_{si} + {s}_{11}^{si} {h}_{p} ) , & ( 7 ) \\ B = \frac{{h}_{si} ( {h}_{si} + {h}_{p} )}{( {s}_{11}^{p} {h}_{si} + {s}_{11}^{si} {h}_{p} )} & ( 8 ) \\ and & \\ K = 4 {s}_{11}^{p} {s}_{11}^{si} {{h}_{si} ( {h}_{p} )}^{3} + 4 {s}_{11}^{p} {{s}_{11}^{si} ( {h}_{si} )}^{3} {h}_{p} + {( {s}_{11}^{p} )}^{2} {( {h}_{si} )}^{4} + & ( 9 ) \\ {( {s}_{11}^{si} )}^{2} {( {h}_{p} )}^{4} + 6 {s}_{11}^{p} {{s}_{11}^{si} ( {h}_{si} )}^{2} {( {h}_{p} )}^{2} & \end{matrix}$$

In these equations the subscripts ‘si’ and ‘p’ denote the elastic silicon layer and the piezoelectric thin film respectively, while d₃₁, s₁₁, h, g, m and l are the piezoelectric coefficient, the compliance, the layer thickness, the gravitational acceleration constant, the mass of the needle and the cantilever length, respectively. The subscripted numbers i and j (i.e. dᵢⱼ, sᵢⱼ, etc.) indicate the direction of the applied electric field and the direction of the resulting normal strain, respectively. The compliance s₁₁ is the reciprocal value of the Young's Modulus E. Equations (5) and (6) depend on two external factors: (1) the passive load (weight) of the microneedle; and (2) the voltage V (or electric field) which is applied to the piezoelectric layer. For small displacements, the vertical deflection is linearly proportional to the electric field E₃=−V/hₚ that is used to electrically stress the piezoelectric layer between its upper and lower surface. A more efficient way to affect the vertical displacement δ is by increasing the length l of the beam.

The lateral force Fₗ produced at the cantilever-tip by applying an electric field is calculated as follows:

$${F}_{l} = \frac{{d}_{31} V w {h}_{s i} ⌊ {{s}_{11}^{s i} ( {h}_{p} )}^{3} + {{s}_{11}^{p} ( {h}_{s i} )}^{3} ⌋}{K} \qquad ( 10 )$$

The vertical force F, which is the force required to penetrate the skin, is calculated multiplying the lateral force Fₗ with the sine of the deflection angle α (see FIG. 4.4(a)). The relationship in Eq. (10) indicates that increasing the horizontal width w increases the actuation force at the tip of the cantilever. The same effect takes place when the applied voltage V is increased.

Table 4.1 illustrates different piezoelectric thin film materials, their piezoelectric coefficient d₃₁, the Young's Modulus E and the compliance s₁₁ [52, 55].

TABLE 4.1
Properties of thin-film piezoelectric materials.

| | d31 | E | s11 |
|---|---|---|---|
| Material | [pm/V] | [MPa] | [1/MPa] |
| Aluminium Nitride (AlN) | −2 | 345 | 0.003 |
| Zinc Oxide (ZnO) | −5 | 123 | 0.008 |
| Polyvinylidene Fluoride (PVDF) | −23 | 3 | 0.33 |
| Lead Zirconium Titanate (PZT) | −170 | 53 | 0.02 |

Lead Zirconium Titanate (PZT) is widely used as a transducer material and finds many applications in the Microactuation-Technology. In terms of large displacement, PZT is preferred because of its very high piezoelectric coefficient d₃₁. The principle disadvantage of using PZT is the complexity associated with its reliable deposition and fabrication [56]. Solving Eq. (5) numerically with beam dimensions of l=4000 um, w=400 um, hᵢₛ=15 um and hₚ=5 um, using the materials PZT and silicon (E=165 GPa, [49]) and applying 60V resulted in a deflection of δ=550 um at the tip of the cantilever. The resulting angle (see Eq. (6)) at the tip was calculated to be α=16° and the resulting force F=45 mN. Scaling down the layer thickness hᵢₛ and hₚ to 7 um and 1 um respectively, no more than 10V are needed to obtain a deflection δ of 500 um but with a resulting force of about 10 mN. A low driving voltage is preferred for a better integration and compatibility with contemporary CMOS technology. The contribution of the mass m_{N} of the microneedle in terms of deflection and force is minimal. In the idle state, the deflection due to the passive load gm_{N} was calculated to be no more than 0.5 um.

It has been shown [57] that optimal efficiency in power occurs, when the silicon layer thickness hᵢₛ is designed about 1.5 to 2 times larger then the piezoelectric layer hₚ. For this particular design however, it has been calculated, that for an optimal force-displacement relationship, the silicon layer has to be between 3 to 7 times thicker then the piezoelectric layer.

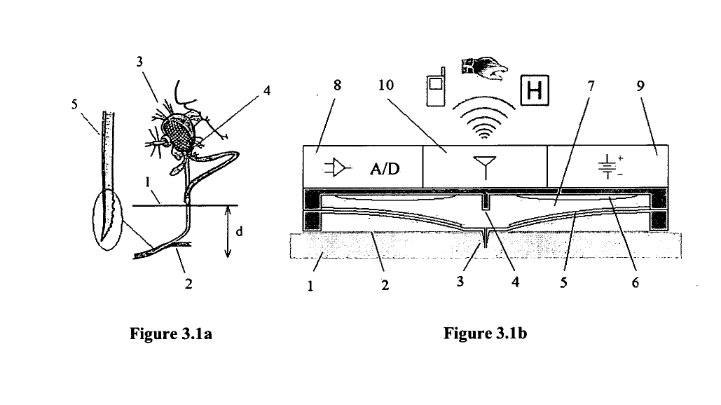

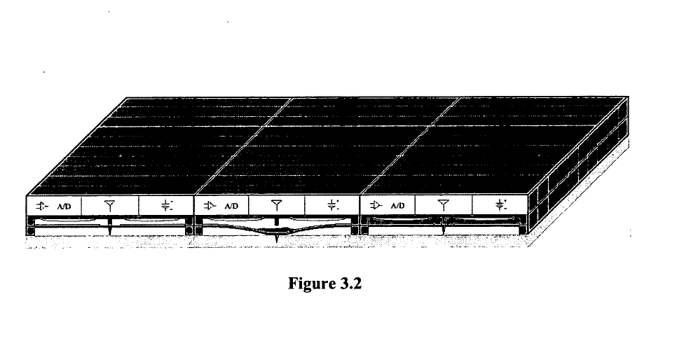

FIG. 4.5 demonstrates the actuation design of the e-Mosquito™. The bridge configuration compared to a single beam configuration features an elegant solution to integrate the blood compartment on top of the microbridge (see FIG. 3.1). This avoids the problematic guiding of the blood from the needle base to a separate blood compartment through a microchannel.

The microbridge (A1) bonds the two actuation beams (A2) together forming a microbridge. The microneedle (see Section 4.1) is implemented on the lower side of the microbridge and moves towards the skin in vertical direction when the benders are actuated. A vertical aperture in the microbridge with the diameter D (same as the microneedle diameter) permits the blood to flow through the needle into the compartment. During the actuation of the two benders, the connector is exposed to longitudinal and perpendicular stress. Therefore, it has to meet the following requirements: (1) it has to be horizontally flexible enough not to inhibit significantly the vertical deflection of the two benders, but on the other hand, (2) it has to withstand the force acting vertically from the microneedle as well as the horizontal stress from the benders.

The ANSYS software program [58] was used in conjunction with SolidWorks [59] to simulate the behavior of the microbridge under structural loading conditions. An ANSYS-based automated Finite Element Analysis (FEA) was performed to generate the results listed below (FIG. 4.6).

The following material properties of silicon [49] were utilized during the analysis: Young's Modulus of 150 GPa, yield stress of 7 GPa, Poisson's ratio of 0.278, and mass density of 2330 kg/m³. The maximal stress measured in the beam was 0.426 GPa which is safely below the yield stress (safety factor of 16.43), and therefore indicates that the beam won't break under a deformation of about 500 um.

Although this section was focused on piezoelectric actuation, it should be mentioned here that magnetic, thermal and electrostatic actuation were explored as well as a solution to actuate the microneedle. Particularly of interest for this application is the thermal actuation principle, being advantageous in (1) the manifestation of large displacements; (2) consuming a low operating voltage; and (3) being relatively straightforward to fabricate. Thermal actuation however has the drawback of operating under high temperatures and is therefore unlikely to be biocompatible when in contact with blood or medications. Other less applicable techniques for the motion-control of the e-Mosquito™ microneedle would be magnetic and electrostatic actuation.

Microsensor

The glucose microsensor for the e-Mosquito™ is an electrochemical transducer in which current is converted from the chemical domain into the electrical domain through oxidation or reduction of at the electrode surface. With the basic transducer being a small metal electrode insulated everywhere except at a specific location on which the chemical reactions take place, several electrochemical analytical and synthetic systems can be implemented. The type of electrode sensor is differentiated on the basis of the electrical parameter that is measured. In the case of glucose measurements, potentiometric and amperometric sensors can be utilized [51]. Current research shows that amperometry is more popular than potentiometry for the design of glucose biosensors [60-64]. This is mainly because the detecting functions of amperometry are based on the usage of an enzyme that has the advantage of fair simplicity, high selectivity, good sensitivity and a large dynamic range [60].

When considering the chemical reactions pertinent to the amperometry scheme, the chemical processes can be accomplished using an external voltage source. Such implementation can reduce the impact of external disturbing factors like oxygen [65]. Using advanced measurement procedures and digital signal processing methods, linear measurable range can be defined for normal subjects and diabetics with blood glucose in the range of 0.17-2.22 mM [66].

Amperometry employs the enzyme glucose oxidase (GOD) to convert the chemical reaction rate into a current. The use of GOD for glucose detection is well known, and is a common biosensor application [51]. An example of an amperometric biosensor is presented in an important study [67], where a polyvinyl alcohol (PVA) matrix was used to apply GOD to a Platinum (Pt) working electrode. A thick-film Silver (Ag) reference electrode was used. In the presence of oxygen, glucose present in the blood is oxidized:
glucose+O₂→gluconolactone+H₂O₂ (11)

The Pt electrode is held at a potential of 0.7V with respect to the Ag electrode, and thus any hydrogen peroxide (H₂O₂) present is oxidized, releasing hydrogen ions and causing a current to flow:
H₂O₂→O₂+2H⁺+2e⁻ (12)

By measuring the current, the glucose concentration can be determined. Referring to Eq. 12, one molecule of glucose being oxidized by a commonly-used two-electrode electrochemical cell results in the emission of 2 free electrons. The induced current Iᵢ can be derived according to the following equation [60]:

$${I}_{i} = n F A D \frac{\partial [ O ]}{\partial x} \qquad ( 13 )$$

where n is the number of electrons transferred in the reaction (valence number), F is the Faraday constant, A is the area of the electrodes, D is the diffusion coefficient of the species of interest, O is the concentration of oxygen and x is the distance between the electrodes.

To define the dimensions of the microsensor of the e-Mosquito™, the dynamic range of the induced current Iᵢ from the chemical reaction on a tiny enzymatic area is considered first. FIG. 4.7 shows the relationship between the induced current Iᵢ and the blood glucose concentration on an area of A=20 mm² [60]. It can be observed, that the dynamic range of the induced current Iᵢ is from 0 to 12.5 uA.

When considering the constraints of the microcircuit design and practical glucose measurements, the enzymatic area cannot be too small. Assuming, that the effective blood-exposed working-electrode (Pt) area of the e-Mosquito™ compartment is in the range of 4×0.06 mm² (FIG. 4.8), the induced current Iᵢ can be linearly scaled down, according to Eq. 13, to a range of 0 to 1.5 μA.

FIG. 4.8 illustrates the main parts of the e-Mosquito™ microsensor. Ion-Sensitive Field Emission Transistors (ISFETs) are envisioned to implement the microsensor electrodes. The wall dimensions of the blood compartment coincide with the length and the width of the microactuator (discussed in Section 4.2) for optimal integration.

Microelectronics

The output signal of the glucose microsensor is a current I approximately in the range between 0 and 1.5 μA. This current has to be conditioned by several electrical stages to be read remotely as a glucose concentration on a display. FIG. 4.9 illustrates in a block diagram these electrical stages.

The rectangular blocks (E01-E14) in FIG. 4.9 represent electrical stages, of which (E01-E04) and (E11-E14) are physically part of the e-Mosquito™ system. The electrical stages (E05-E10) belong physically to a remote controlling device (i.e. watch, cell phone, personal digital assistant, etc.).

A single e-Mosquito™ cell is selected by decoding, and actuation is initiated, resulting in the acquisition of a blood sample, which is sensed by the glucose microsensor. The current I resulting from the microsensor operation is converted in the first stage (E01) into a voltage vₛ. The sensor voltage vₛ is very small, and has therefore to be amplified and conditioned in the second stage (E02). In this stage a comparator is included as well, comparing the obtained voltage to a predetermined minimal level to ensure that a meaningful glucose reading has been indeed provided by the microsensor. The resulting analog voltage Vₛ is converted into a digital signal in stage three (E03). The RF transmitter, denoted as stage four (E04), captures the digital signal (0-1) to transmit it wirelessly (maximal range of 5 m) to the RF receiver (E05) of the remote device. In the remote device, a data logger (E06) sends the signal to a microcontroller (E07), from which the processed signal is delivered to a display (E08) and is finally read as a glucose concentration. The eighth stage (E08) completes the blood sampling process and closes the glucose-monitoring loop. In addition, the microcontroller output is utilized to close the insulin control loop by controlling an external insulin infusion pump (E09) or by simply transmitting control sequence through the RF transmitter (E10) back to the on-chip microelectronic control of the e-Mosquito™ to actuate dedicated drug-delivery cells in it.

Upon completion of the blood sampling and glucose concentration reading, the remote microcontroller (E07) decides when to issue another actuation signal to a different e-Mosquito™ cell. Two scenarios are possible: (a) if the current glucose-reading has been successful, the next actuation is preprogrammed to occur according to a specific protocol designed for the individual patient utilizing the e-Mosquito™ patch; or (b) in case of unsuccessful readout from the particular e-Mosquito™ cell, another actuation of a new cell is immediately initiated. It should be emphasized once again that each individual e-Mosquito™ cell is actuated only once and re-actuations are not envisioned.

The actuation signal is wirelessly transmitted (maximal range of 5 m) to the RF receiver (E11), which is placed in the e-Mosquito™. An on-chip microcontroller (E12) receives the incoming feedback signal and controls the actuation of the next e-Mosquito™ cell. The microcontroller (E12) is remotely programmable to control not only the actuation, but also the transmission of the conditioned analog voltage Vₛ representing the sensed blood glucose level to the analog-to-digital converter (E03). FIG. 4.10 details the on-chip operation of an individual e-Mosquito™ cell and its interrelation with adjacent cells.

The serially-obtained analog signals from different e-Mosquito™ cells are multiplexed to the input of the A/D converter (E03) via a wired-OR setup facilitated by CMOS switches (E17), controlled by the logical combination of three distinct sources: (1) the inverted microcontroller signal (E15) initiating the actuation; (2) a timing signal generated by the microcontroller (E12) and initiated after the completion of the actuation of the [k−1]^{th} e-Mosquito™ cell; and (3) a comparator signal originating from the conditioning circuit (E02), indicating that the measured voltage has reached a given predetermined level thus ensuring that the blood sampling from the particular cell has been successful. These three signals are grouped in an AND logic (E16) to close the normally opened CMOS switch (E17) of the k^{th} e-Mosquito™ cell, thus providing an input to the A/D converter (E03). At the same time the respective CMOS switches of all other e-Mosquito™ cells remain opened, thus implementing the wired-OR multiplexing.

Ideally, all on-chip e-Mosquito™ microelectronics is implemented using Very Large Scale Integration (VLSI) level design. However, discrete electronic implementation is also possible, particularly for feasibility studies.

The electronic implementation and control of the e-Mosquito™ drug delivery system operates similarly, with the major difference of having the microactuator inserting the microneedle into the skin to inject insulin or a medical remedy instead of sampling blood (FIG. 4.11).

Microbattery

For the e-Mosquito™, electrical energy is required to operate the microactuator, the microsensor, the microelectronics and the microtransceiver. MEMS pose a genuine challenge for energy management. With the present technology, energy must be supplied from outside sources, such as a battery or DC power supply and the power loss during the transmission into the circuit can be very large [68]. For MEMS which are intended to be autonomous or remote (i.e., physically unattached to a power source), the power supply has presented the biggest challenge [69, 70]. The battery is usually the largest contributor to both the overall weight and the volume of the MEMS device [70].

Microscopic energy storage is possible in a variety of forms. Authors of an important paper [70] surveyed a range of possibilities for powering MEMS, including mechanical energy, magnetic or electrical fields, chemical energy, radiative energy and even fission reactors (fission releases neutrons, radiation and energy in the form of heat). They concluded that the most promising energy storage options for MEMS devices would be microscopic batteries and electrical/magnetic field devices (such as capacitors). Capacitors have a long life, but their energy densities are low. Fuel cells are possible, but the storage and delivery of fuel is problematic. Realistically, batteries can provide adequate power and energy for the present application.

The battery for the e-Mosquito™ needs to meet the following criteria: (1) the process used to implement the battery must be a part of a multifunctional, integrated device development process; (2) the battery must not have excessive volume or weight; (3) it must store high energy and power density and be capable of delivering short pulses of high power for the actuation; (4) in order to manage changes in temperature, pressure and humidity without major performance degradation, the battery must be very robust.

In order to determine the approximate total power consumption of the e-Mosquito™ and choose its optimal energy source, the following functional building blocks of the e-Mosquito™ are modeled separately as a power consuming component: (1) the microactuator; (2) the microsensor; the (3) analog conditioning stage (E01-E03); (4) the RF transceiver (E04&E11); (5) the controlling microelectronics (E12); and (6) the voltage booster (E14).

For the microactuation a static voltage supply V_{A} equal to 10V is assumed. In order to calculate the power P_{A1} consumed by one PZT actuation beam on the bridge, the following equation is applied:

$${P}_{A1} = \frac{{V}_{A}^{2}}{{R}_{A}} \qquad ( 14 )$$

where R_{A} is the resistance of the piezoelectric material (PZT). The resistance R_{A} can be calculated applying the following formula:

$${R}_{A} = \frac{ρ {h}_{p}}{l w} \qquad ( 15 )$$

where ρ=10.3⁸ Ωm [71] is the resistivity of PZT, hₚ=5 um, l=4000 um and w=400 um are the thickness, length and the width of the PZT plate, respectively (see FIG. 4.4(b)). Solving these equations numerically, and multiplying the power consumption P_{A1} by the factor of two, since there are two PZT plates on the bridge, the power consumption for one single actuation is P_{A}=0.126 uW. With this information, the current I_{A} that flows vertically between the upper and the lower surface of the PZT plate can be calculated as follows:

$${I}_{A} = \frac{{P}_{A}}{{V}_{A}} \qquad ( 16 )$$

The resulting current of a single actuation is hence I_{A}=12.6 nA. It is assumed that the e-Mosquito™ patch (see Section 3) has 180 independently actuated cells and therefore the total amount of current I_{A180} that flows resulting from actuation, is calculated as 2.27 uA. The capacity Q_{A} resulting from the actuation can be formulated with the following equation:
Q_{A}=I_{A}t_{A} (17)
with t_{A}=4 s being the time for actuation. The total capacity Q_{A} resulting from an actuation equals to 0.455 uAh, which is relatively low.

For the quantification of the capacity Q_{S} consumed by the e-Mosquito™ microsensor, the current Iₛ resulting from a single sensor, is assumed to be equal to its induced current Iᵢ (see Section 4.3). The time tₛ utilized to measure the glucose concentration is assumed to be around 60 s [72] and therefore the total capacity Q_{S} consumed for glucose sensing can be calculated applying again Eq. 17 as 0.81 mAh.

The analog signal conditioning stage (E01-E03) foreseen for the e-Mosquito™ is driven with 3V, utilizes a supply current I_{Con} of 1 mA during its operational time t_{Con}, which is presumed to be around 20 s per e-Mosquito™ cell. The standby current I_{Cstby} used by the conditioner while the device is inactive, is denoted as 10 μA. The duration is t_{STBY} and consequently the total capacity Q_{C} consumed by the analog signal conditioner (E01-E03) can be estimated to be around 2.60 mAh.

The RF transceiver (E04 and E11) envisioned for the e-Mosquito™ is operated with 3V, and utilizes a supply current I_{Ton} of 5.3 mA during its operational time t_{Ton}, which is assumed to be around 5 s per e-Mosquito™ cell. The standby current I_{Tstby} consumed by the transceiver while the device is idle is about 1.7 μA. This duration t_{STBY} is assumed to be a week or 168 h and therefore the total capacity Q_{T} consumed by the RF transceiver (E04&E11) can be estimated to be around 1.61 mAh.

The microcontroller (E12) envisioned for the e-Mosquito™ is operated with 3V, and utilizes a supply current I_{Mon} of 3 mA during its operational time t_{Mon}, which is assumed to be around 10 s per e-Mosquito™ cell. The standby current I_{Mstby} consumed by the microcontroller while the device is idle, is about 9 μA. Again, the duration equals to t_{STBY} and hence the total capacity Q_{M} consumed by the microcontroller (E12) can be estimated to be approximately 3.17 mAh.

The voltage booster (E14) foreseen for the e-Mosquito™ is driven with 3V, and utilizes a supply current I_{Bon} of 50 mA during its activate time t_{Bon} being 4 s per e-Mosquito™ cell (equal to actuation time t_{A}). The standby current I_{Bstby} during the time t_{STBY} is estimated at about 10 μA. The total capacity Q_{B} consumed by the voltage booster (E14) is therefore estimated to be in the range of 10.68 mAh.

The microelectronic block is considered to be with 70% efficiency (a rather conservative estimation), calculated from the total energy consumption. Microbatteries which are as small as 29×22 mm² in area, 0.44 mm in height and 0.6 g in weight have become recently commercially available [73]. These cells have a nominal voltage of 3V and a capacity of 25 mAh. The energy density reaches up to 110 Wh/kg. Based on the above considerations, these microbatteries are quite suitable for the e-Mosquito™ application.

System Integration

The building blocks of the e-Mosquito™ that were outlined in Section 4 have to be assembled and incorporated into a complete integrated microsystem. The nomenclature is kept constant during the entire section to improve the readers understanding. The x-y-z triad at the bottom left of the 3D figures illustrate the actual position in space of the device referring to the x-y-z coordinate system.

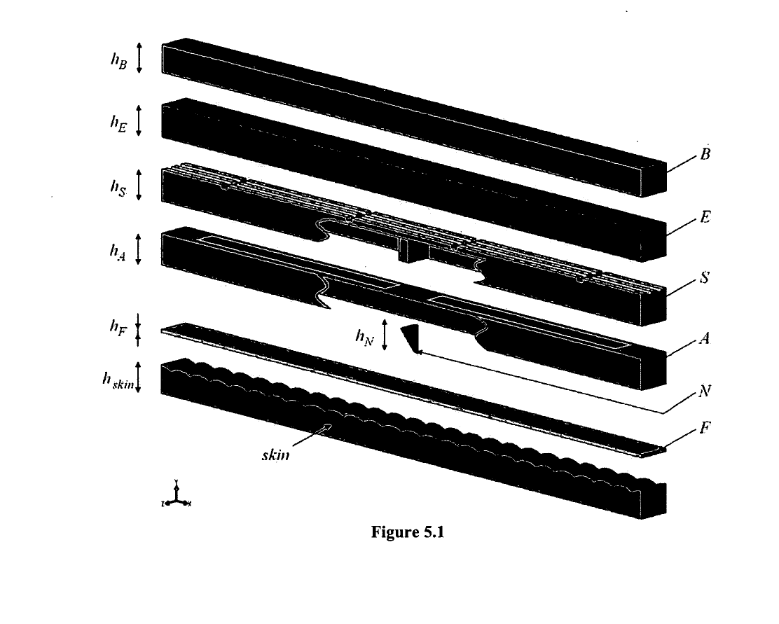

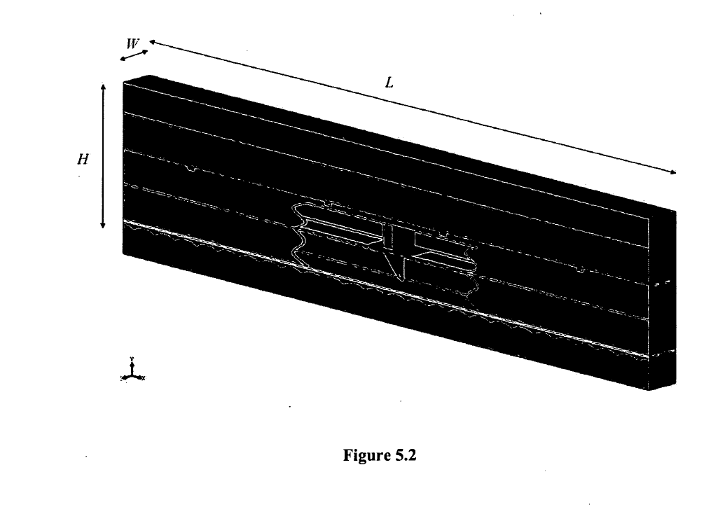

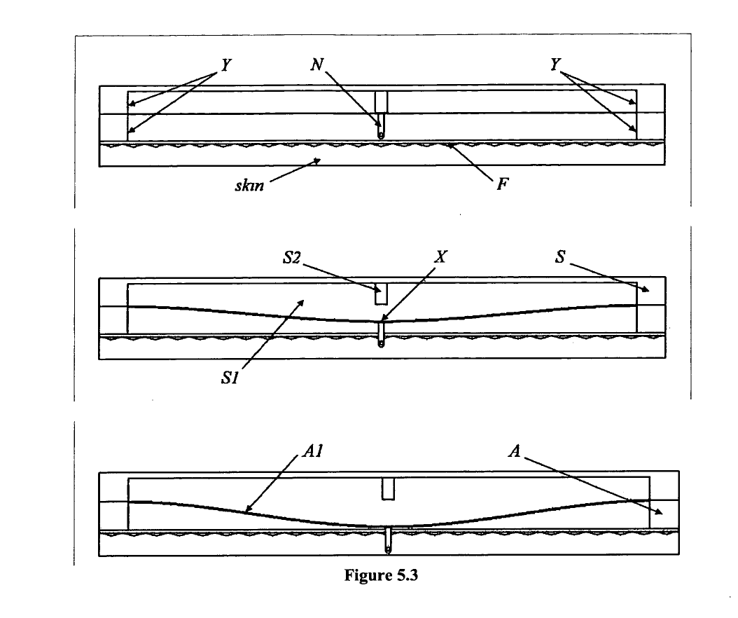

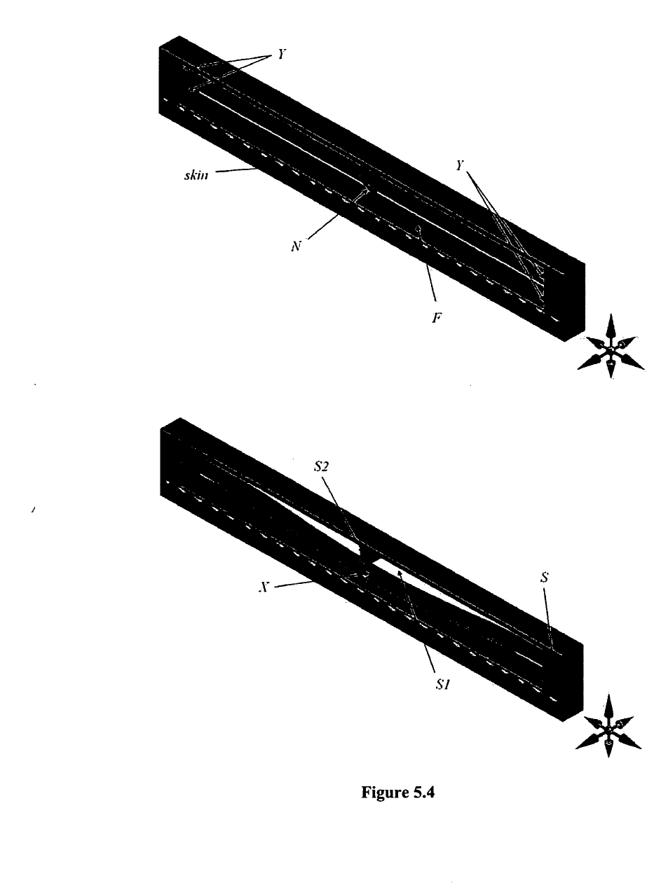

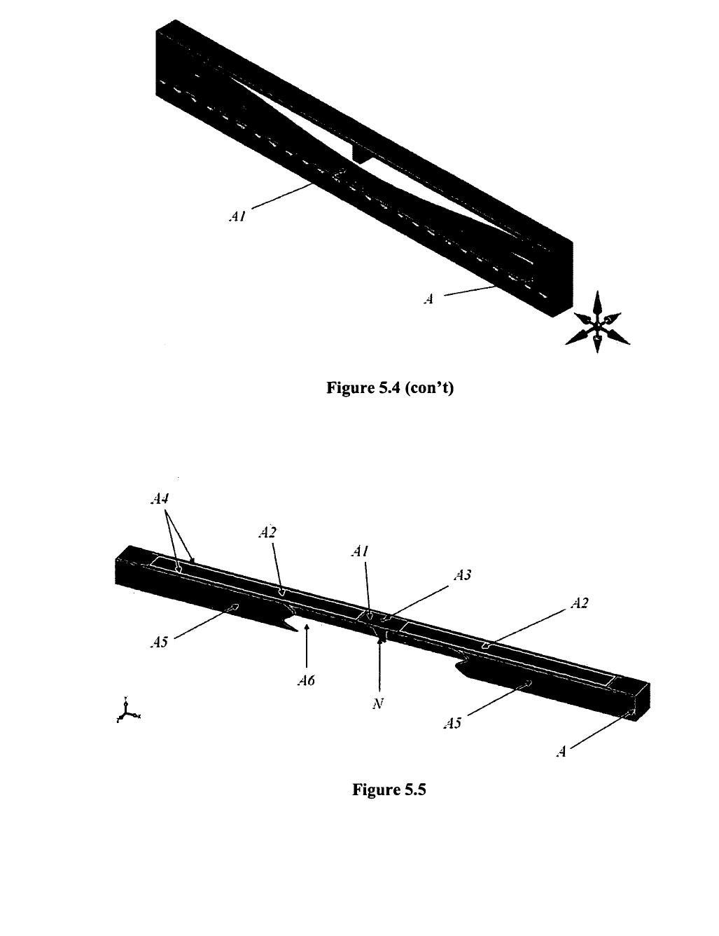

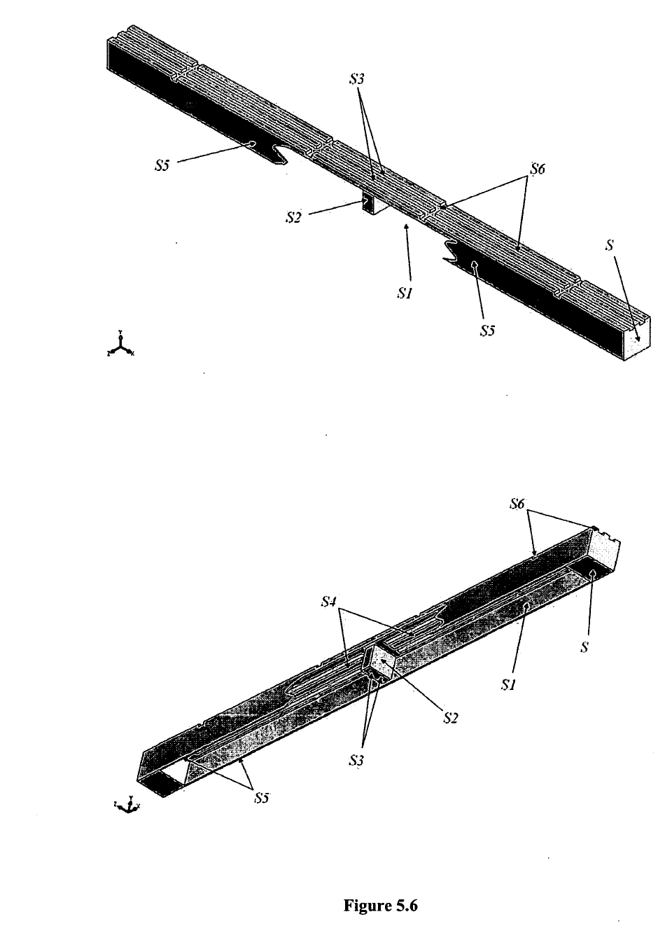

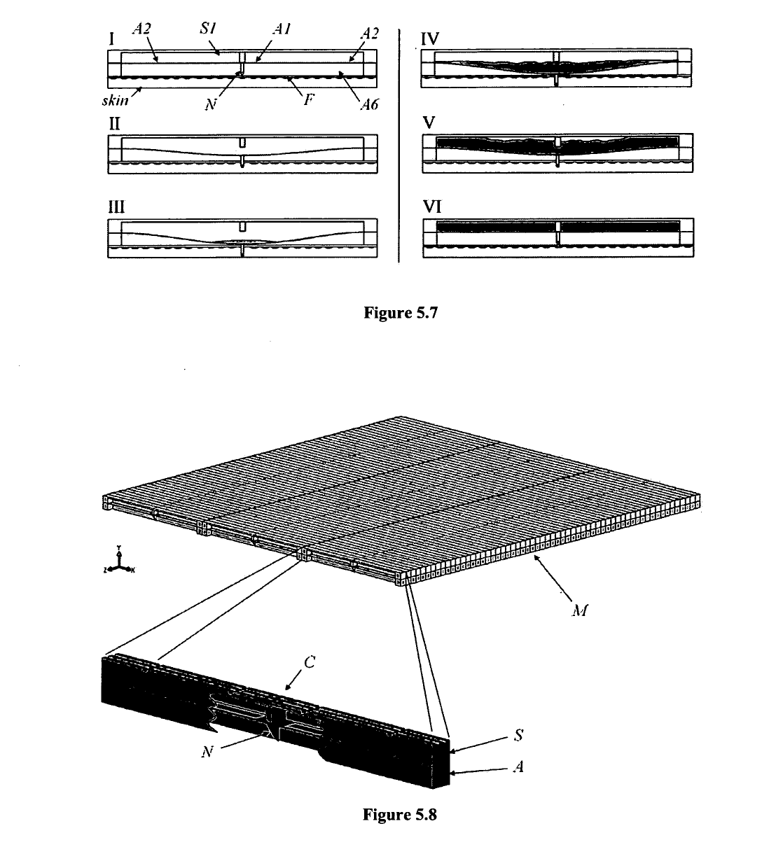

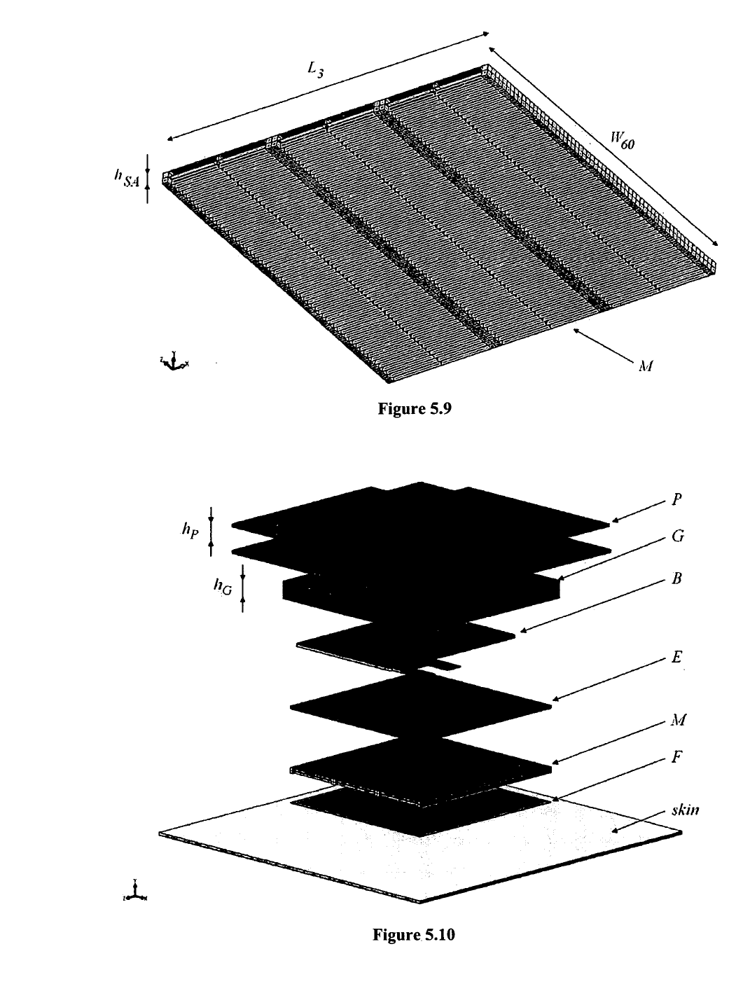

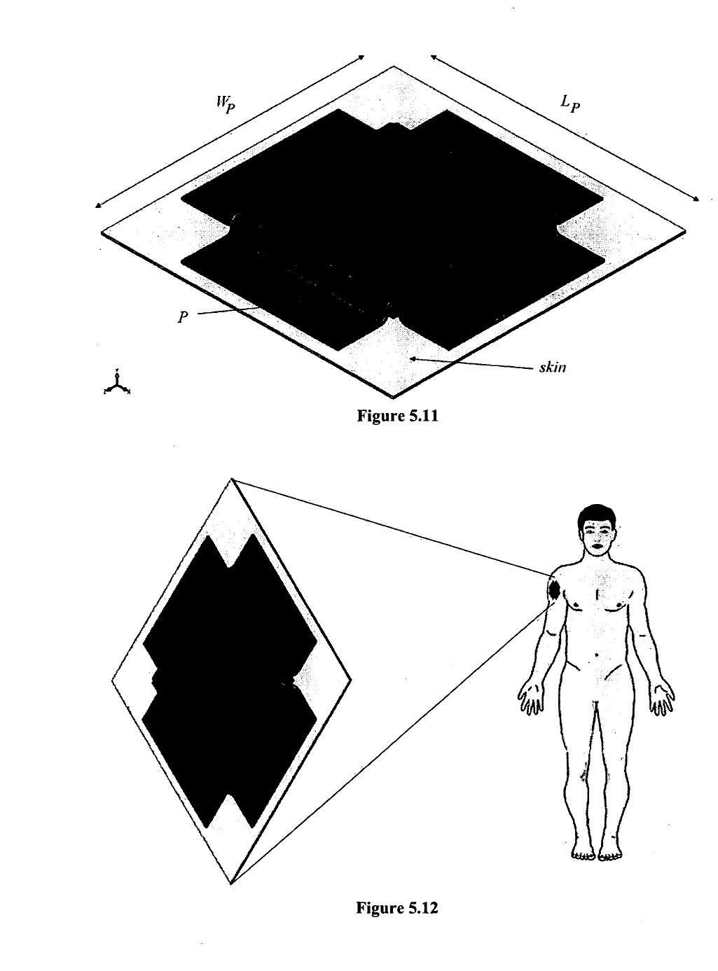

FIG. 5.1 shows an exploded view of the e-Mosquito™ identifying its most important components. The microsensor structure (S) and the microactuator structure (A) are partly dissected at the side walls to better illustrate their design. The microelectronic block (E) as well as the microbattery (B) are only laid out symbolically, for more details refer to Sections 4.4 and 4.5, respectively. The antiseptic layer (F) is adhesively coated to promote secure adhesion to the skin surface. The purpose of this attachment is to inhibit any bacterial or viral contaminants to enter the e-Mosquito™ environment resulting in (a) probable inadequate measurements of the microsensor, (b) potential clogging of any micro orifices (i.e. microneedle orifice, the pressure gaps, etc) in the e-Mosquito™ structure, and (c) latent transcutaneous infection of the subject carrying the device. The thickness (h_{F}) of the adhesive film is in the range of 0.1 mm. The heights of the microactuator (h_{A}) and the microsensor (h_{S}) structure are 0.5 mm each. The height of the microbattery (h_{B}) is 0.44 mm (see Section 4.5) and the height of the microelectonic building block (h_{E}) is in the range of 0.5 mm if a VLSI design is implemented, or 3 mm if assembled using discrete components.

FIG. 5.2 presents a compact version of FIG. 5.1 and elucidates how the assembled e-Mosquito™ looks like. The total height (H) of the e-Mosquito™ is the sum of the building block heights (hⱼ) and is consequently in the range of 2 mm for a VLSI version, or about 6 mm for a discrete component version.

The first step of assembling the e-Mosquito™ is to integrate the microneedle, the microactuator and the microsensor into one working part. FIG. 5.3 presents a 2D view of these three elements and illustrates clearly the blood sampling action performed by the e-Mosquito™.

In order to model the actuation and subsequent penetration of e-Mosquito™ as accurate as possible, an ANSYS simulation environment was developed with the following parameters: The microneedle (N) as well as the microbridge (A1) were associated with the material properties of silicon (see Section 4.2). The skin and the adhesive and antiseptic layer (F) were modeled with the mechanical properties of human skin [74] (Young's Modulus: 333 MPa; Yield Stress: 1.7 MPa; Poisson's Ratio: 0.3; and Mass Density: 1100 kg/m³). The sidewalls (Y) of the microactuator structure and the microsensor structure respectively, were mechanically fixed for the purposes of the simulation. A vertical force of 25 mN was applied (X) on top of the microbridge. FIG. 5.4 shows a three-dimensional simulation model of the e-Mosquito™.

The results of this simulation show that (a) the microneedle successfully penetrates skin to a depth of 500 μm without fracturing; and (b) the safety factor for the entire assembly is in the range of 23 and hence withstands the large displacement.

FIGS. 5.3 and 5.4 illustrate the principle of operation of the e-Mosquito™. The actuation duration t_{A} is time-controlled and is in the range of 4 s. It is estimated, based on the volume V=1.44 μl of the blood compartment and the microfluidic calculations outlined in Section 4.1, resulting in a flow rate through the microneedle of Q=0.341 μl/s.

During the microactuation step, blood enters into the blood compartment (S1). In order to avoid blood leak through the microbridge (A1) and the actuator side wall (A5) into the actuator compartment (A6), the gaps between the microbridge and the side wall, labeled side gaps (A4) have to be very small. In order to benefit from the surface tension of the blood (see Section 4.1) between the actuator side-wall (A5) and the microbridge, the side-gaps have to be 5 um or smaller, to avoid any leakage of blood from the compartment.

FIG. 5.5 illustrates a three-dimensional view of the microactuator structure (A) and the partially visible actuator compartment (A6). The actuator orifice (A3) has the same diameter (D) (see Section 4.1) as the microneedle and is precisely aligned with it to facilitate continuous blood flow from the microneedle (N) into the blood compartment (S1).

The purpose of the microvalve (S2) is: (a) to maintain a sealed and sterile blood compartment (S1) that is isolated from the surrounding environment throughout the idle period; and (b) to maintain the air pressure equilibrium between the blood compartment and its surroundings during the actuation time tₐ. This is accomplished with the two pressure gaps (S3) illustrated in FIG. 5.6.

The tiny dimensions of the pressure gaps (S3) are in the range of 5 μm allowing the air to pass, but inhibiting the blood in the compartment (S1) to leak (refer to the blood-related microfluidic calculations in Section 4.1). The two pressure gaps connect the blood compartment (S1) and the air-channels (S6) during the period of actuation. The air-channels have a width of 100 μm, a depth of 20 μm and are implemented on the top of the microsensor structure (S). The channels are sealed on top with the microelectronic building block (E), but are left open at the edges to interface to an air channel network through the entire e-Mosquito™ matrix. This air channel network provides an atmospheric pressure environment and therefore facilitates to the elimination of pressure gradients during the actuation.

FIG. 5.7 summarizes in six distinct instances (I-VI) the principle of operation of the e-Mosquito™ blood sampling procedure. Instance (I) illustrates an e-Mosquito™ cell while it is idle. This state lasts as long as there is no electrical voltage applied to its PZT microactuators (A2). When actuation is administrated (II) under the control of an on-chip microcontroller, the microbridge bends (refer to Section 4.2) and drives the microneedle (N) vertically down towards the skin by piercing through the adhesive & antiseptic layer (F). The antiseptic layer minimizes infections as well as irritations at the skin surface of the subject and seals the actuator compartment (A6) and therefore the entire e-Mosquito™ from its surrounding environment while the device is in the idle state (I). In instance (III), the microbridge (A1) reaches its maximal displacement positioning the tip of the microneedle (N) at a depth of approximately 500 um underneath the surface of the skin. Capillary forces and interstitial pressure (see Section 4.1) facilitate blood flow into the e-Mosquito™ blood compartment (S1). At instance (IV), the voltage continues to be applied to the PZT microactuator (A2), and the microneedle (N) is maintained at its maximal penetration depth. When the compartment (S1) is about to be filled up with blood (V), the actuation voltage is removed, the microbridge bends towards its initial position (I) and hence the microneedle (N) is pulled out of the skin. The actuation time tₐ between instances (II) and (V) is approximately 4 s (refer to Section 4.1), which depends on the blood flow rate through the microneedle. In instance (VI), the microbridge (A1) and the microneedle (N) are again in their initial position (I) leaving the blood compartment filled with blood, thus concluding the e-Mosquito™ blood sampling process.

A similar process can be utilized for drug delivery, with the actuation being applied on the top of the compartment, which would in this case contain the drug to be delivered, rather then being used for blood collection.

The next level of integration and assembly of the e-Mosquito™ is introduced as follows. The microactuator structure (A), microneedle (N) and microsensor structure (S) are brought together to a structure “single e-Mosquito™ cell” (C). This single e-Mosquito™ cell is then assembled into an entire network forming the complete “e-Mosquito™ matrix” (M). FIG. 5.8 illustrates the single e-Mosquito™ cell (C) and the entire e-Mosquito™ matrix (M).

The dimensions of the e-Mosquito™ matrix (M) depend mainly on the dimensions of a single e-Mosquito™ cell. The height (h_{SA}) of the matrix equals to the sum of the height of the microsensor structure (h_{S}), and the height of the microactuator structure (h_{A}), respectively. The dimensions of the e-Mosquito™ matrix are shown in FIG. 5.9.

The length and width of the e-Mosquito™ matrix are denoted as L₃ and W₆₀ respectively, given that three single e-Mosquito™ cells (C) are assembled along L₃ and 60 cells are assembled along its width W to build a matrix of 180 individually actuated e-Mosquito™ cells (C). Substituting the dimensions numerically, the e-Mosquito™ matrix has a square area of 900 mm² with a length (L₃) of 30 mm, a width (W₆₀) of 30 mm and a height (h_{SA}) of 1 mm. Each e-Mosquito™ is individually controlled by the microelectronic block (E) and powered by the microbattery (B). An example of the individual e-Mosquito™ actuation is illustrated in FIG. 3.2.

The e-Mosquito™ matrix as shown in FIG. 5.9, is integrated into an “e-Mosquito™ patch” (see FIG. 5.10), which includes the antiseptic film (F), the microelectronics (E), the microbattery (B), the housing (G) and the Band-Aid (P). FIG. 5.10 illustrates the exploded view of the e-Mosquito™ patch.

The microelectronics (E) is shown as a building block of the e-Mosquito™ patch and includes all relevant electronic components (i.e. microcontroller, signal conditioner, A/D converter, RF transceiver, high voltage charge pump, etc.) outlined in Section 4.4. The design of the microelectronic block (E) can be (1) either entirely VLSI-based, (2) with discrete components, or (3) a hybrid of the two. The dimensions of the microelectronics block (E) are more then sufficient to incorporate all the necessary electronic circuitry to actuate and control the e-Mosquito™ patch.

The characteristics and dimensions of the microbattery (B) are outlined in Section 4.5. The adhesive Band-Aid (P) and the housing (G) are building blocks that haven't been discussed yet. The housing is essentially a hollow box made out of durable plastic material with the purpose to protect the very delicate MEMS device inside. It has a wall thickness of approximately 1 mm to withstand the rough environment on the skin of a human being. The height h_{G} of the housing is consequently constituted by the height H of the e-Mosquito™ (refer to FIG. 5.2) plus the thickness of the housing wall (=1 mm) resulting in h_{G}, which numerically equals to 3 mm for the VLSI version, or 7 mm for the discrete-component version. The height h_{P} of the Band-Aid (P) above the skin surface equals to the total height of the e-Mosquito™ patch above the skin surface and depends on the thickness of the used Band-Aid, it can though be roughly assumed that h_{P} is in the range of 3.5 mm, or 7.5 mm for the discrete-component version. A three-dimensional view of the e-Mosquito™ patch being attached to the skin is given in FIG. 5.11.

With the e-Mosquito™ matrix (M) being 30×30 mm², the dimensions of the entire e-Mosquito™ patch is in the maximal range of 50×50 mm². An example showing possible attachment of the e-Mosquito™ on a person is illustrated in FIG. 5.12.

The region of attachment of the e-Mosquito™ patch should be an area which has high blood circulation (i.e. skeletal muscle) and where the circulation comes close to the skin surface. A possible region that meets these requirements is the deltoid muscle which encompasses the shoulder bones.

Fabrication

The fabrication and assembly process of the e-Mosquito™ is divided into the fabrication steps (I-V). Step (I) outlines the microfabrication of the e-Mosquito™ microactuator (A) including the microneedle (N). The manufacturing of the e-Mosquito™ microsensor (S) is illustrated in step (II), while step (III) demonstrates the fabrication of the e-Mosquito™ microelectronics (E). The bonding of the three building blocks (AN, S, and E) is discussed in step (IV) and the final assembly with the remaining parts including the microbattery (B), the adhesive layer (F), the housing (G) and the Band-Aid (P) are outlined in step (V). The wide arrows in the figures illustrate, whether (1) material is either removed (etched) from the wafer (arrow departs from the wafer), or (2) material is added (deposited) onto the wafer (arrow arrives to the wafer). The e-Mosquito™ figures shown in this section illustrate the microfabrication process and are not to be scaled.

The next deposited layer (I4) is again metal (Pt/Au), which forms the upper electrode of the piezoelectric actuator. On top of this electrode, an insulating material (silicon nitride) is deposited (I5) to protect from the high voltage that is applied to the upper electrode. Step (I6) illustrates a reactive ion etching (RIE) process, in which (1) the orifice of the microneedle (N) is pre-etched (arrow in the center) and (2) the actuator side walls (A5) are separated from the microbridge (A1). Up to step (I6) all the fabrication procedures take place on the immediate surface of the wafer. In step (I7), the bottom of the wafer is bulk-etched using DRIE, resulting in a microbridge and a square microstructure with an orifice. Since the microneedle orifice is pre-etched in step (I6), the same etch-depth for the orifice and the bridge can be utilized in (I7). In order to obtain the sharp tip of the microneedle (N), anisotropic etching is employed in step (I8) resulting in the final design marked as (AN).

To summarize, procedure (I) is a combination of surface-micromachining (I1-I6) and bulk-micromachining (I7-I8), applied to manufacture the e-Mosquito™ microactuator (A) and the microneedle (N).

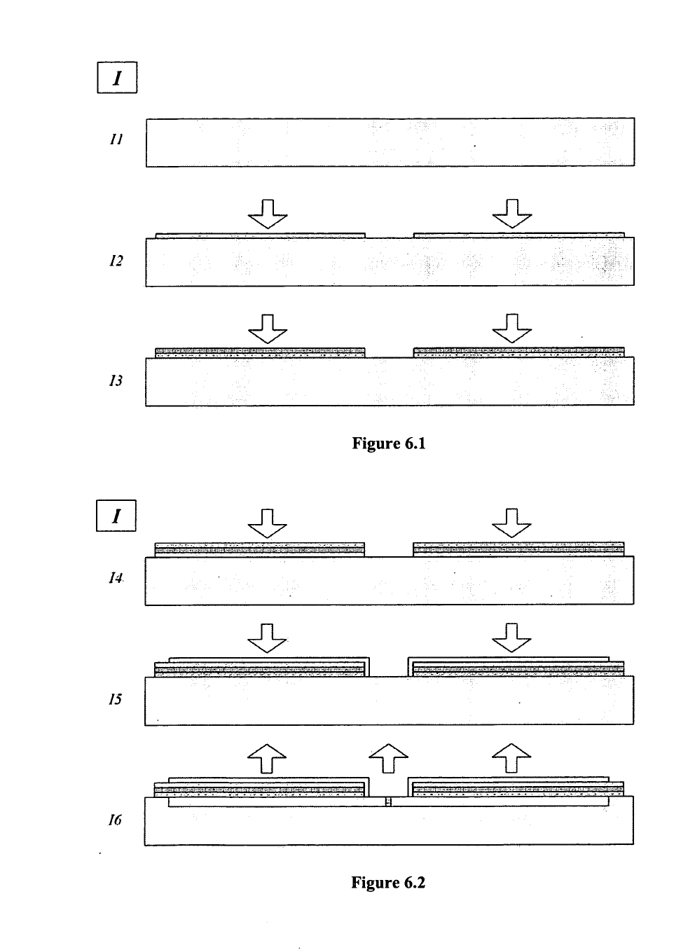

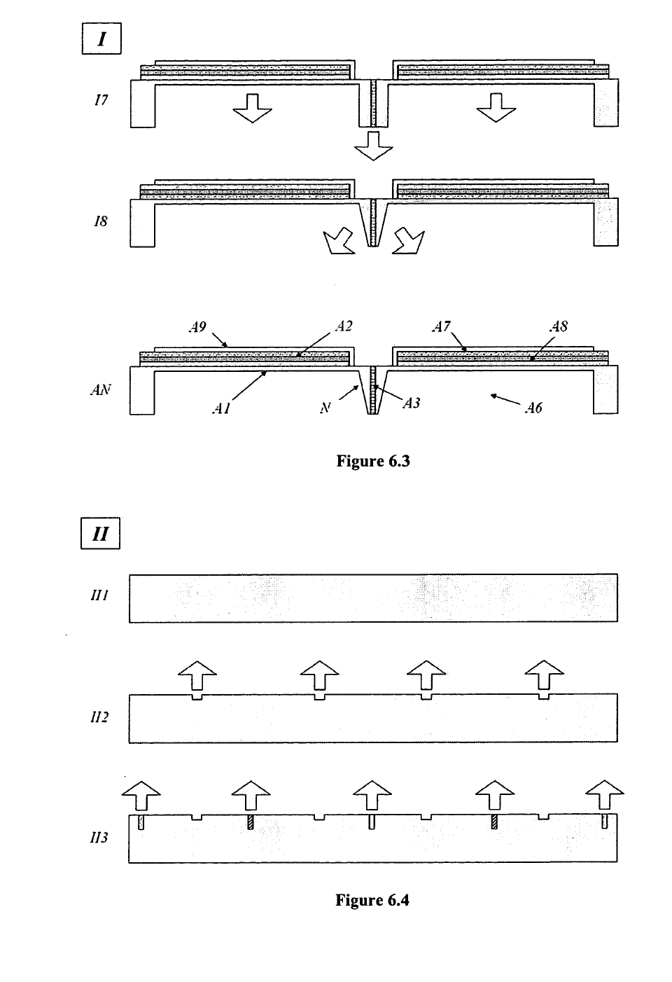

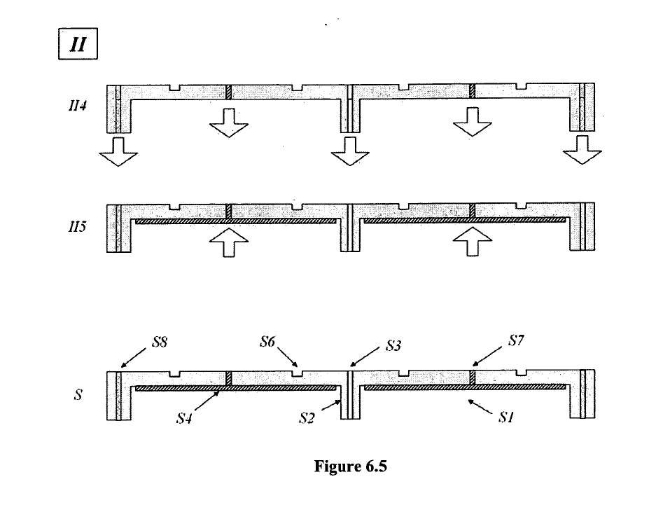

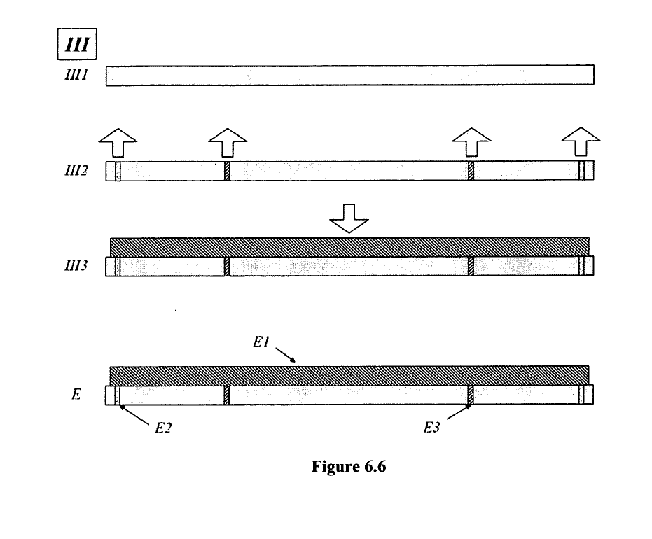

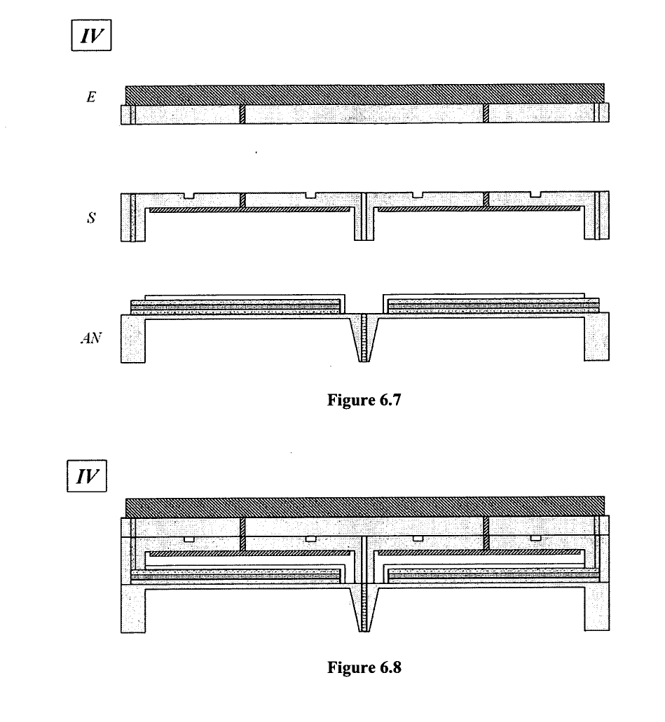

The fabrication of the e-Mosquito™ microsensor (S) is outlined in the next procedure (II) and illustrated in FIGS. 6.4 and 6.5. Again, a single crystal silicon (SCS) wafer with a thickness of 500 um is utilized (III), for the manufacturing of the microsensor structure (S). The first step is the removal (RIE) of Silicon (II2) to form the future micro air channels (S6) on top of the structure (S). The second step (II3) is to pre-etch to a certain depth the electrical connection pathways to the microsensor electrodes (S4) and to the microactuator electrodes (A2).

The purpose of the holes pre-etched in (II3) and completely etched from the opposite side of the wafer (II4) are to connect electrically the e-Mosquito™ microelectronics (E) with the microsensor structure (S) and the microactuator structure (A). The outermost two holes connect the microactuator electrodes (A2), and the two holes nearest to the center electrically connect the microsensor electrodes (S4). The hole etched through the vertical centre of the structure forms the pressure gap (S3). In step (II4), a DRIE process is used to bulk micromachine the e-Mosquito™ blood-compartment (S1), as well as the e-Mosquito™ microvalve (S2). The deposition of the microsensor electrodes (S4) is further signalized in step (II5), and (S) illustrates the complete e-Mosquito™ microsensor structure (S).

The fabrication of the e-Mosquito™ microelectronics (E) is the next fabrication procedure to be tackled (III), and is illustrated in FIG. 6.6. For the microelectronic VLSI chip, a thin silicon wafer (200 um) is chosen, since (1) a high aspect ratio is not an issue for microelectronic circuits; (2) the distance of the micro connection lines (micro buses) has to be short to avoid loses and noise; and (3) the overall height of the e-Mosquito™ patch has to be kept as small as possible. Four feed through holes are etched (III2), which facilitate electrical contact to the microelectronic components (E) with the microactuator (A) and the microsensor (S). Step (III) illustrates the sub-assembly of the VLSI system onto the wafer. The schematic of the e-Mosquito™ microelectronics (E) is included at the bottom of FIG. 6.6.

The micromachining of the three most important building blocks (AN, S, and E) of the e-Mosquito™ system have been outlined. The next procedure (IV) is their assembly to form an integrated microsystem. FIG. 6.7 illustrates the assembly of the three e-Mosquito™ building blocks.

The three structures are assembled and fixtured using a low temperature wafer-bonding process. The cross-sectional view of the final (AN, S, E) assembly is shown in FIG. 6.8.

The three-wafer fabrication procedure (I-IV), outlined above is advantageous in many ways. One of the most important benefits is its compatibility with the implementation of the e-Mosquito™ matrix (see Section 5). A silicon wafer containing a matrix of microsensors (S) is aligned and bonded on top of a wafer holding a matrix of microactuators (A), with the same characteristics as outlined in procedure (I) and (II). A third wafer containing the microelectronics (E) (see process (III)) is then implemented on top of the wafer containing the matrix of microsensor structures (S), similarly to the outlined procedure (IV).

Immaterial modifications may be made to the embodiments described here without departing from the invention. Claim elements are understood to refer to the embodiments disclosed and their equivalents now known or hereinafter developed. The use of the word “comprising” or the indefinite article “a” in the claims is not intended to exclude other elements being present.

### REFERENCES

[1] R. Ogawa and T. Togawa, “Attempts at monitoring health status in the home,” presented at 1st Annual International Conference on Microtechnologies in Medicine and Biology, 2000.

[2] R. A. Peura, “Blood Glucose Biosensors—a Review,” presented at IEEE Cast Studies in Medical Instrument Design, 1991.

[3] L. Eriksson and I. Kanno, “Blood sampling devices and measurements,” Medical Progress through Technology, vol. 17, pp. 249-257, 1991.

[4] T. Kuriyama, “Non-invasive blood glucose monitoring,” presented at Proceedings of the 1995 8th International Conference on Solid-State Sensors and Actuators and Eurosensors IX. Part 1 (of 2), Jun. 25-29, Stockholm, Sweden, 1995.

[5] H. M. Heise, “Technology for non-invasive monitoring of glucose,” presented at Proceedings of the 18th Annual International Conference of the IEEE Engineering in Medicine and Biology Society, Part 5 (of 5), Oct. 31-Nov. 3, Amsterdam, Netherlands, 1996.

[6] K. Xu, Q. Li, Z. Lu, and J. Jiang, “Fundamental study on non-invasive blood glucose sensing,” Journal of X-Ray Science and Technology, vol. 10, pp. 187-197, 2002.

[7] D. A. Gough, J. C. Armour, and D. A. Baker, “Implementation of the implantable glucose sensor,” presented at Proceedings of the 15th Annual International Conference of the IEEE Engineering in Medicine and Biology Society, Oct. 28-31, San Diego, Calif., USA, 1993.

[8] V. Poitout, D. Moatti-Sirat, and G. Reach, “Development of a glucose sensor for glucose monitoring in man: The disposable implant concept,” Clinical Materials, vol. 15, pp. 241-246, 1994.

[9] K. W. Johnson, J. J. Mastrototaro, D. C. Howey, R. L. Brunelle, P. L. Burden-Brady, N. A. Bryan, C. C. Andrew, H. M. Rowe, D. J. Allen, B. W. Noffke, W. C. McMahan, R. J. Morff, D. Lipson, and R. S. Nevin, “In vivo evaluation of an electroenzymatic glucose sensor implanted in subcutaneous tissue,” Biosensors & Bioelectronics, vol. 7, pp. 709-714, 1992.

[10] S. Chandrasekaran, J. D. Brazzle, and A. B. Frazier, “Surface micromachined metallic microneedles,” Journal of Microelectromechanical Systems, vol. 12, pp. 281-288, 2003.

[11] P. Griss and G. Stemme, “Side-opened out-of-plane microneedles for microfluidic transdermal liquid transfer,” Journal of Microelectromechanical Systems, vol. 12, pp. 296-301, 2003.

[12] D. V. McAllister, M. G. Allen, and M. R. Prausnitz, “Microfabricated microneedles for gene and drug delivery,” Annual Review of Biomedical Engineering, vol. 2, pp. 289-313, 2000.

[13] L. W. Lin and A. P. Pisano, “Silicon-processed microneedles,” Journal of Microelectromechanical Systems, vol. 8, pp. 78-84, 1999.

[14] M. R. Prausnitz, “Drug delivery by electrical, ultrasonic, and microneedle disruption of biological barriers,” Abstracts of Papers of the American Chemical Society, vol. 219, pp. 176-MEDI, 2000.

[15] B. L. Stoeber, “Fluid injection through out-of-plane microneedles,” presented at 1st Annual International Conference on Microtechnologies in Medicine and Biology, 2000.

[16] J. G. E. B. Gardeniers, J. W.; de Boer, M. J.; Yeshurun, Y.; Hefetz, M.; van't Oever, R.; van den Berg, A., “Silicon micromachined hollow microneedles for transdermal liquid transfer,” presented at The Fifteenth IEEE International Conference on Micro Electro Mechanical Systems, 2002.

[17] S. M. Chandrasekaran, A. B. Frazier, “Autonomous microneedle system for biochemical analysis,” presented at 12th Innational Conference on TRANSDUCERS, Solid-State Sensors, Actuators and Microsystems, 2003.

[18] B. Stoeber, “An Integrated MEMS Syringe for Advanced Drug Delivery: Design, Fabrication and Fluid Mechanics of Suspension Flow through Microneedle Arrays,” in Mechanical Engineering. Berkeley: University of California, 2002, pp. 180.

[19] M. R. Prausnitz, “Microneedle drug delivery device,” in U.S. Pat. No. 6,611,707, 2003.

[20] K. Oka, S. Aoyagi, Y. Arai, Y. Isono, G. Hashiguchi, and H. Fujita, “Fabrication of a micro needle for a trace blood test,” presented at Transducers'01 Eurosensors XV, Jun. 10-14, Munich, 2002.

[21] A. Hodges, “Method and device for sampling and analyzing interstitial fluid and whole blood samples,” in U.S. Pat. No. 6,612,111: Lifescan, Inc., 2003.

[22] LifeScan, “OneTouch® UltraSmart™ Blood Glucose Monitoring System, Johnson & Johnson,” 2002.

[23] V. V. Yuzhakov, “Apparatus and method for using an intracutaneous microneedle array,” in U.S. Pat. No. 6,256,533: The Procter & Gamble Company, 2001.

[24] F. F. Sherman, “Apparatus and method for manufacturing an intracutaneous microneedle array,” in U.S. Pat. No. 6,312,612, 2001.

[25] M. J. N. Cormier, “Device and method for enhancing transdermal flux of agents being sampled,” in U.S. Pat. No. 6,537,264, 2003.

[26] J. P. Sopp, “Methods of sampling body fluid,” in U.S. Pat. No. 6,624,882, 2003.

[27] M. J. Botich, “Fluid collection device with captured retractable needle,” in U.S. Pat. No. 6,540,732, 2003.

[28] G. R. Mauze, “Physiological fluid extraction with rapid analysis,” in U.S. Pat. No. 6,375,627: Agilent Technologies, Inc., 2002.

[29] D. L. Swendson, “Closed one-handed blood sampling system,” in U.S. Pat. No. 6,224,561: Edwards Lifesciences Corporation, 2001.

[30] R. I. Connelly, “Wearable, self-contained drug infusion device,” in U.S. Pat. No. 6,589,229, 2003.

[31] R. J. Pettis, “Microneedle-based pen device for drug delivery and method for using same,” in United States Patent Application No. 20030050602: Becton, Dickinson and Company, 2003.

[32] S. T. Cho, “Microneedles for minimally invasive drug delivery,” in United States Patent Application No. 20020193754: Abbott Laboratories, 2002.

[33] J. G. Aceti, “Autonomous, ambulatory analyte monitor or drug delivery device,” in United States Patent Application No. 2003153900: Sarnoff Corporation, 2003.

[34] A. Angel, “Microneedle Transdermal Transport Device,” in World Intellectual Property Organization WO/03037404 A1, 2002.

[35] A. N. Clements, The physiology of mosquitoes. Oxford: Pergamon Press, 1963.

[36] S. F. Chandrasekaran, A. B., “Characterization of surface micromachined hollow metallic microneedles,” presented at IEEE The Sixteenth Annual International Conference on Micro Electro Mechanical Systems, MEMS-03 Kyoto, 2003.

[37] J. Brazzle, I. Papautsky, and A. B. Frazier, “Micromachined needle arrays for drug delivery or fluid extraction—Design and fabrication aspects of fluid coupled arrays of hollow metallic microneedles,” IEEE Engineering in Medicine and Biology Magazine, vol. 18, pp. 53-58, 1999.

[38] P. M. Wang, M. Cornwell, and M. R. Prausnitz, “Microneedles for transdermal delivery of insulin and extraction of interstitial fluid for glucose monitoring,” Faseb Journal, vol. 17, pp. A556-A556, 2003.

[39] M. A. Shikida, M.; Ishibara, Y.; Yamaura, N.; Ando, T.; Sato, K.; Asaumi, K., “Fabrication of pen-shaped microneedle structure by using non photolithographic pattern transfer,” presented at 12th International Conference on TRANSDUCERS, Solid-State Sensors, Actuators and Microsystems, 2003.

[40] S. S. Sang-Jun Moon, “Fabrication of microneedle array using inclined LIGA process,” presented at 12th International Conference on TRANSDUCERS, Solid-State Sensors, Actuators and Microsystems, 2003.

[41] P. S. Griss, “Novel, side opened out-of-plane microneedles for microfluidic transdermal interfacing,” presented at The Fifteenth IEEE International Conference on Micro Electro Mechanical Systems, 2002.

[42] S. Davis, M. R. Prausnitz, “The mechanics of microneedles,” presented at EMBS/BMES Conference, 2002.

[43] S. Davis, M. G. Allen, “Fabrication and characterization of laser micromachined hollow microneedles,” presented at 12th International Conference on TRANSDUCERS, Solid-State Sensors, Actuators and Microsystems, 2003.

[44] S. M. Henry, D. V.; Allen, M. G.; Prausnitz, M. R., “Micromachined needles for the transdermal delivery of drugs,” presented at The Eleventh Annual International Workshop on Micro Electro Mechanical Systems, MEMS 98, 1998.

[45] S. Henry, D. V. McAllister, M. G. Allen, and M. R. Prausnitz, “Microfabricated microneedles: A novel approach to transdermal drug delivery (vol 87, pg 922, 1998),” Journal of Pharmaceutical Sciences, vol. 88, pp. 948-948, 1999.

[46] D. V. K. McAllister, S.; Patel, P. N.; Mayberry, J. L.; Allen, M. G.; Prausnitz, M. R., “Microneedles for transdermal delivery of macromolecules,” presented at BMES/EMBS Conference, 1999.

[47] D. V. McAllister, M. G. Allen, and M. R. Prausnitz, “Microfabricated microneedles for gene and drug delivery,” Annual Review of Biomedical Engineering, vol. 2, pp. 289-313, 2000.

[48] P. M. C. Wang, M. G.; Prausnitz, M. R., “Effects of microneedle tip geometry on injection and extraction in the skin,” presented at EMBS/BMES Conference, 2002.

[49] K. E. Petersen, “Silicon as a Mechanical Material,” Proceedings of the IEEE, vol. 70, pp. p 420-457, 1982.

[50] F. Martini, Fundamentals of anatomy & physiology, 5th ed. Upper Saddle River, N.J.: Prentice Hall, 2001.

[51] G. T. A. Kovacs, Micromachined transducers sourcebook. Boston: WCB/McGraw-Hill, 1998.

[52] M. J. Madou, Fundamentals of microfabrication: the science of miniaturization, 2nd ed. Boca Raton, Fla.: CRC Press, 2002.

[53] D. B. Wood, J. S.; Harris, A. J., “Actuators and their mechanisms in microengineering,” in Engineering Science and Education Journal, vol. 7, 1998, pp. 19-27.

[54] J. G. Smits and W.-s. Choi, “The constituent equations of piezoelectric heterogeneous bimorphs,” IEEE Transactions on Ultrasonics, Ferroelectrics, and Frequency Control, vol. 38, pp. 256-270, 1991.

[55] eFunda, “Online Reference for Engineers, http://www.efunda.com.”

[56] K. Westra, “Personal Communication on Fabrication.” NanoFabLab Director, University of Alberta, Edmonton, Canada, 2003.

[57] J. G. C. Smits, W.-S.; Cooney, T. K., “The efficiency to convert electrical to mechanical energy of a piezoelectric on silicon bimorph micromotor,” presented at Ultrasonics Symposium, IEEE Proceedings, 1991.

[58] ANSYS, “Software Program, Version 7, http://www.ansys.com,” Ansys Inc.

[59] Solidworks, “Software Program, Version 2003, http://www.solidworks.com,” SolidWorks Corporation.

[60] O. T.-C. W. Chen, S.; Chii-Wann Lin; Yao-Chou Lu, “A medical microsensor for blood glucose monitoring,” presented at IEEE International Symposium on Circuits and Systems, ISCAS '97, 1997.

[61] M. Sudoh, A. Mori, H. Fujimura, and H. Katayama, “Development of novel micro-sensor of disposable chips with oxygen enrichment by pre-electrolysis for monitoring blood glucose,” Electrochimica Acta: Proceedings of the 1999 2nd International Symposium on Electrochemical Microsystems Technologies—Electrochemical Applications of Microtechnology, vol. 44, pp. 3839-3848, 1999.

[62] J.-H. Kim, B.-G. Kim, J.-B. Yoon, E. Yoon, and C.-H. Han, “A new monolithic microbiosensor for whole blood analysis,” Sensors and Actuators, A: Physical, vol. 95, pp. 108-113, 2002.

[63] J. Z. Zhu, J. F. Xie, D. R. Lu, G. X. Zhang, and Z. R. Zhang, “Micromachined glucose sensor and K+ ISE based on containment array,” Sensors and Actuators, B: Chemical; IMCS-7: 7th International Meeting on Chemical Sensors, Jul. 27-Jul. 30 1998, vol. 65, pp. 157-159, 2000.

[64] J. Perdomo, H. Hinkers, C. Sundermeier, W. Seifert, O. M. Morell, and M. Knoll, “Miniaturized real-time monitoring system for L-lactate and glucose using microfabricated multi-enzyme sensors,” Biosensors and Bioelectronics, vol. 15, pp. 515-522, 2000.

[65] N. Martens, A. Hindle, and E. A. H. Hall, “Assessment of mediators as oxidants for glucose oxidase in the presence of oxygen,” Biosensors & Bioelectronics, vol. 10, pp. 393-403, 1995.

[66] J. Popp, A. Silber, C. Braeuchle, and N. Hampp, “Sandwich enzyme membranes for amperometric multi-biosensor applications: improvement of linearity and reduction of chemical cross-talk,” Biosensors & Bioelectronics, vol. 10, pp. 243-249, 1995.

[67] H. Hinkers, C. Sundermeier, R. Luerick, F. Walfort, K. Cammann, and M. Knoll, “Amperometric microelectrode array in containment technology,” Sensors and Actuators, B: Chemical; Proceedings of the EUROSENSORS VIII Conference. Part 2 (of 4), Sep. 25-28 1994, vol. B27, pp. 398-400, 1995.

[68] R. M. H. LaFollette, J. N.; Humble, P., “Microfabricated secondary batteries for remote, autonomous, electronic devices,” presented at The Sixteenth Annual Battery Conference on Applications and Advances, 2001.

[69] K. B. Bult, “Low power systems for wireless microsensors,” presented at International Symposium on Low Power Electronics and Design, 1996.

[70] P. B. B.-V. Koeneman, I. J.; Wood, K. L., “Feasibility of micro power supplies for MEMS,” Microelectromechanical Systems, Journal of, vol. 6, pp. 355-362, 1997.

[71] L. Wu, “The DC resistivity of modified PZT ceramics,” J. Phys. C: Solid State Phys., vol. 16, pp. 2822-2832, 1983.

[72] D. Gough, K. Kreutz-Delgado, and T. Bremer, “Frequency characterization of blood glucose dynamic,” Ann Biomed Eng, vol. 31(1), pp. 91-7, 2003.

[73] VARTA, LFP 25: Lithium Manganese Dioxide Primary Microbattery: VARTA Microbattery GmbH, 2003.

[74] F. M. Hendriks, “A numerical-experimental method to characterize the non-linear mechanical behaviour of human skin,” Skin Research and Technology, vol. 9, pp. 274-283, 2003.

1. (canceled)

2. An automated closed-loop real-time microsystem for fluid sampling, multicomponent analysis from a biological body, and pharmaceutical agent delivery to the said body, comprising of a single autonomously powered integrated microchip platform, the system including: (a) at least one microneedle, (b) at least one microactuator, (c) at least one miniature compartment, (d) at least one microsensor, and (e) a microelectronic system, for the purpose of fluid sampling, multicomponent analysis, pharmaceutical agent delivery, diagnostic aid, and disease control.

3. (canceled)

4. (canceled)

5. (canceled)

6. The microsystem of claim 2 wherein the microneedle is at least individually addressable, controlled, and actuated by the microactuator along the longitudinal direction of the microneedle.

7. (canceled)

8. (canceled)

9. (canceled)

10. (canceled)

11. The microsystem of claim 2 wherein the actuator has at least the shape of a cantilever beam (microbeam), being clamped (fixed) on one end and free to deflect on the distal end.

12. The microsystem of claim 2 wherein the actuator has at least the shape of a microbeam that is attached at both distal ends and thus forming a clamped beam (microbridge).

13. The microsystem of claim 2 wherein no physical displacement of the actuator occurs as long as it is not being electrically, thermally, magnetically stimulated and or actuated.

14. The microsystem of claim 2 wherein the microneedle is attached to the cantilever beam at the location where the maximal displacement of the actuator occurs.

15. The microsystem of claim 2 2 wherein the base of the microneedle is integrated with the actuator.

16. The microsystem of claim 2 wherein the maximal displacement of the actuator is time controlled (actuation time).

17. (canceled)

18. The microsystem of claim 2 wherein the compartment is conformed by the microactuator (at the bottom), by the microsensor (at the top) and the compartment walls.

19. The microsystem of claim 2 wherein the compartment is in fluid communication with the hole at the base of the microneedle and other fluidic channels and secondary sample compartments.

20. The microsystem of claim 2 wherein the compartment is a chamber with a limited volume.

21. The microsystem of claim 2 wherein the compartment is manufactured out of a biocompatible material such as silicon, glass and plastic.

22. The microsystem of claim 2 wherein the internal wall of the compartment is coated with at least one biocompatible material to minimize coagulation of fluid comprised primarily of blood.

23. The microsystem of claim 2 wherein the compartment contains a sensor operable to determine when the fluid completely fills the miniature compartment such that the increase of the accumulating fluid may be terminated.

24. The microsystem of claim 2 wherein the ceiling of the compartment holds a protrusion acting as a primary microvalve.

25. The microsystem of claim 2 wherein the primary microvalve is a valve which inhibits a liquid transfer through the microneedle during the idle state of the microactuator.

26. The microsystem of claim 2 wherein the primary microvalve is a valve which separates the fluid compartment into two separate fluid compartments and allows differential measurements of the fluid samples in each compartment.

27. The microsystem of claim 2 wherein the protrusion contains at least one microchannel acting as a secondary microvalve.

28-39. (canceled)

40. The microsystem of claim 2 wherein at least one microchannel is implemented horizontally between the bottom of the microelectronics and the outside wall of the ceiling of the miniature compartment claimed in claim 34.

41-70. (canceled)

71. The microsystem of claim 2 mounted on a disposable patch attached to the said body with an adhesive antiseptic layer.
